(19) United States
(12) Patent Application Publication
(10) Pub. No.: US 2016/0067377 A1
(43) Pub. Date: Mar. 10, 2016

(54) Stem Cell Seeded Natural Substrates and Methods Relating Thereto

(71) Applicant: AlloSource, Centennial, CO (US)

(72) Inventors: Yaling Shi, Larkspur, CO (US); Simon Bogdansky, Littleton, CO (US); Brent L. Atkinson, Littleton, CO (US); Brian Dittman, Lenexa, KS (US); Reginald L. Stilwell, Parker, CO (US)

(73) Assignee: ALLOSOURCE, Centennial, CO (US)

(21) Appl. No.: 14/940,798

(22) Filed: Nov. 13, 2015

Related U.S. Application Data
(63) Continuation-in-part of application No. 12/612,583, filed on Nov. 4, 2009, now Pat. No. 9,192,695.
Application No. 12/612,583 is a continuation-in-part of application No. 12/965,335, filed on Dec. 10, 2010.
Application No. 12/965,335 is a continuation-in-part of application No. 12/612,583, filed on Nov. 4, 2009, now Pat. No. 9,192,695.
Application No. 12/612,583 is a continuation-in-part of application No. 14/207,220, filed on Mar. 12, 2014, now abandoned.
(60) Provisional application No. 61/116,484, filed on Nov. 20, 2008.
(60) Provisional application No. 61/285,463, filed on Dec. 10, 2009.
(60) Provisional application No. 61/116,484, filed on Nov. 20, 2008.
(60) Provisional application No. 61/790,412, filed on Mar. 15, 2013.

(51) Int. Cl. A61L 27/24; C12N 5/0775; A61L 27/50; A61L 27/36; A61L 27/38

(52) U.S. Cl. CPC A61L 27/24; A61L 27/3687; A61L 27/3834; A61L 27/50; C12N 5/0667; A61L 2430/34; A61L 2400/18; C12N 2533/54; C12N 2537/00

(57) ABSTRACT

This disclosure provides compositions for treating tissue injuries comprising a tissue-derived substrate and mesenchymal stem cells adhered thereto, as well as methods of making and using such compositions. The tissue-derived substrates include bone, cartilage, and collagen matrix.

### CROSS-REFERENCE TO RELATED APPLICATIONS

This application is a continuation-in-part application of U.S. application Ser. No. 12/612,583, filed Nov. 4, 2009, which claims the benefit of priority to U.S. Provisional Application No. 61/116,484, filed Nov. 20, 2008. The entire contents of each of these applications are incorporated herein by reference in their entirety.

This application is also a continuation-in-part application of U.S. application Ser. No. 12/965,335, filed Dec. 10, 2010, which claims the benefit of priority to U.S. Provisional Application No. 61/285,463, filed Dec. 10, 2009, and which is a continuation-in-part application of U.S. application Ser. No. 12/612,583, filed Nov. 4, 2009, which claims the benefit of priority to U.S. Provisional Application No. 61/116,484, filed Nov. 20, 2008. The entire contents of each of the these applications are incorporated herein by reference in their entirety.

In addition, this application is a continuation-in-part application of U.S. application Ser. No. 14/207,220, filed Mar. 12, 2014, which claims benefit of priority of U.S. Provisional Application No. 61/790,412, filed Mar. 15, 2013. The entire contents of each of these applications are incorporated herein by reference in their entirety.

### BACKGROUND

Regenerative medicine deals with the process of replacing, engineering or regenerating human cells, tissues or organs to restore or establish normal function. Some regenerative medicine approaches focus on the implantation of tissues, scaffolds, stem cells, or a combination thereof into injury or defect sites in a patient.

Injuries to hard or soft tissues, such as bone, skin, muscle, connective tissue, or vascular tissue, are common occurrences. In some instances, minor soft or hard tissue injuries are able to self-repair without any outside intervention, but frequently the extent of an injury is severe enough, or the capacity of the soft or hard tissue to self-repair is limited enough, that surgical intervention is required. Surgery to repair a hard or soft tissue injury generally entails implanting or applying a biocompatible material that is meant to replace the missing or defective tissue (for example, using a graft to replace a torn tendon/ligament or bone). However, even with surgical intervention, the process of repairing or reconstructing the injured soft tissue can be slow or incomplete.

Allografts may be combined with stem cells. This generally requires a significant amount of tissue processing and cellular processing prior to seeding the allograft substrate. In some instances, regenerative medicine requires an abundant source of human adult stem cells that can be readily available at the point of care. Allografts seeded with living cells may provide better surgical results.

Stem cells have been shown to be useful in promoting wound healing and the repair of injuries to soft tissues such as tendons and ligaments. See, e.g., Yin et al., Expert Opin. Biol. Ther. 10:689-700 (2010); Hanson et al., Plast. Reconstr. Surg. 125:510-6 (2010); and Cha and Falanga, Clin. Dermatol. 25:73-8 (2007). Stem cells have also been used to promote soft tissue reconstruction, for example using stem cell-seeded small intestinal submucosa to promote bladder reconstitution and meniscus reconstruction. Chung et al., J. Urol. 174:353-9 (2005); Tan et al., Tissue Eng. Part A 16:67-79 (2010). Similarly, stem cells have also been used to promote bone reconstruction. For example, adipose-derived stem cells (ASCs), which can be obtained in large quantities, have been utilized as cellular therapy for the induction of bone formation in tissue engineering strategies.

### BRIEF SUMMARY

Provided are methods of making an allograft composition for treating a tissue injury, the method comprising: (a) providing a cell suspension comprising mesenchymal stem cells and non-mesenchymal stem cells derived from tissue obtained from a cadaveric donor; (b) seeding the cell suspension onto a tissue scaffold derived from tissue obtained from the cadaveric donor; (c) incubating the tissue scaffold seeded with the cell suspension under conditions suitable for adhering the mesenchymal stem cells to the tissue scaffold to form a seeded scaffold; and (d) rinsing the seeded scaffold to remove the non-adherent cells from the seeded scaffold, thereby forming the allograft composition comprising the tissue scaffold with mesenchymal stem cells adhered thereto.

In one aspect, there is provided a method of combining mesenchymal stem cells with a bone substrate, the method comprising obtaining tissue having the mesenchymal stem cells together with unwanted cells; processing (e.g., digesting) the tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In one aspect, there is provided a method of combining mesenchymal stem cells with an osteochondral allograft, the method comprising obtaining adipose tissue or other tissue having the mesenchymal stem cells together with unwanted cells; processing (e.g., digesting) the adipose tissue or other tissue to form a cell suspension having the mesenchymal stem cells and the unwanted cells; adding the cell suspension with the mesenchymal stem cells to seed the osteochondral allograft so as to form a seeded osteochondral allograft; and allowing the cell suspension to adhere to the osteochondral allograft for a period of time to allow the mesenchymal stem cells to attach.

In one embodiment, there is disclosed a method of combining mesenchymal stem cells with cartilage, the method comprising obtaining the mesenchymal stem cells from adipose tissue or other tissue containing mesenchymal stem cells of a cadaveric donor; obtaining the cartilage from the same cadaveric donor; adding the mesenchymal stem cells to seed the cartilage so as to form a seeded cartilage; and allowing the cell suspension to adhere to the mesenchymal stem cells and the cartilage for a period of time to allow the mesenchymal stem cells to attach.

In one aspect, this disclosure provides compositions for treating a soft tissue injury in a subject. In some embodiments, the composition comprises a collagen matrix and mesenchymal stem cells adhered to the collagen matrix, wherein the mesenchymal stem cells are derived from a tissue processed to form a cell suspension comprising mesenchymal stem cells and non-mesenchymal stem cells that is seeded onto the collagen matrix, and wherein the mesenchymal stem cells are not cultured ex vivo after formation of the cell suspension and prior to seeding of the cell suspension on the collagen matrix.

In another aspect, this disclosure provides methods of treating a soft tissue injury in a subject. In some embodiments, the method comprises contacting a composition as described herein (e.g., a composition comprising a collagen matrix and mesenchymal stem cells adhered to the collagen matrix, wherein the mesenchymal stem cells are derived from a tissue processed to form a cell suspension comprising mesenchymal stem cells and non-mesenchymal stem cells that is seeded onto the collagen matrix, and wherein the mesenchymal stem cells are not cultured ex vivo after formation of the cell suspension and prior to seeding of the cell suspension on the collagen matrix) to the site of the soft tissue injury.

In another aspect, this disclosure provides methods of making a composition for treating a soft tissue injury. In some embodiments, the method comprises: (a) processing (e.g., digesting) a tissue to form a cell suspension comprising mesenchymal stem cells and non-mesenchymal stem cells; (b) seeding the cell suspension onto a collagen matrix; (c) incubating the collagen matrix seeded with the cell suspension under conditions suitable for adhering the mesenchymal stem cells to the collagen matrix; and (d) removing the non-adherent cells from the collagen matrix.

In another aspect, provided is a method of making an allograft composition for treating a soft tissue injury, the method comprising: (a) providing a cell suspension comprising mesenchymal stem cells and non-mesenchymal stem cells derived from tissue obtained from a cadaveric donor; (b) seeding the cell suspension onto an acellular collagen matrix derived from tissue obtained from the cadaveric donor; (c) incubating the acellular collagen matrix seeded with the cell suspension under conditions suitable for adhering the mesenchymal stem cells to the acellular collagen matrix to form a seeded matrix; and (d) rinsing the seeded matrix to remove the non-adherent cells from the seeded matrix, thereby forming the allograft composition comprising the acellular collagen matrix with mesenchymal stem cells adhered thereto.

In other aspects, products made by such methods are provided, as are methods of treatment using such products.

### DEFINITIONS

As used herein, the term “soft tissue” refers to a tissue that connects, supports, or surrounds organs and structures of the body, and which is not bone. Examples of soft tissues include, but are not limited to, tendon tissue, ligament tissue, meniscus tissue, muscle tissue, skin tissue, bladder tissue, and dermal tissue.

As used herein, the term “collagen matrix” refers to a biocompatible scaffold comprising collagenous fibers (e.g., collagen I) that provides a structural support for the growth and propagation of cells. In some embodiments, a collagen matrix is a biological tissue that has been harvested from a subject (e.g., a human or non-human animal). Examples of collagen sources include, but are not limited to, skin, dermis, tendon, ligament, muscle, amnion, meniscus, small intestine submucosa, or bladder. In some embodiments, the collagen matrix is from anatomical soft tissue sources (e.g., skin, dermis, tendon, or ligament) and not from bone or articular cartilage. In some embodiments, the collagen matrix primarily comprises type I collagen rather than type II collagen.

As used herein, the term “mesenchymal stem cell” refers to a multipotent stem cell (i.e., a cell that has the capacity to differentiate into a subset of cell types) that can differentiate into a variety of cell types, including osteoblasts, chondrocytes, and adipocytes. Mesenchymal stem cells can be obtained from a variety of tissues, including but not limited to bone marrow tissue, adipose tissue, muscle tissue, birth tissue (e.g., amnion, amniotic fluid, or umbilical cord tissue), skin tissue, bone tissue, and dental tissue.

The term “reduce immunogenicity” or “reduced immunogenicity” refers to a decreased potential to stimulate an immunogenic rejection in a subject. In some embodiments, a collagen matrix as described herein is treated to reduce its immunogenicity (i.e., decrease its potential to stimulate an immunogenic rejection in a subject in which the treated collagen matrix is implanted or topically applied) relative to a corresponding collagen matrix of the same type that has not been treated. The term “non-immunogenic,” as used with reference to a collagen matrix, refers to a collagen matrix which does produce a detectable immunogenic response in a subject.

The terms “decellularized” and “acellular,” as used with reference to a collagen matrix, refer to a collagen matrix from which substantially all endogenous cells have been removed from the matrix. In some embodiments, a decellularized or acellular collagen matrix is a matrix from which at least 70%, 75%, 80%, 85%, 90%, 91%, 92%, 93%, 94%, 95%, 96%, 97%, 98%, 99% or more of endogenous cells have been removed (e.g., by a decellularization treatment), relative to a corresponding collagen matrix of the same type which has not been subjected to removal of endogenous cells (e.g., has not been subjected to a decellularization treatment).

Decellularization can be quantified according to any method known in the art, including but not limited to measuring reduction in the percentage of DNA content in a treated collagen matrix relative to an untreated collagen matrix or by histological staining. In some embodiments, a decellularized or acellular collagen matrix has a DNA content that is reduced by at least 50%, 60%, 70%, 80%, 90% or more as compared to an untreated collagen matrix.

The term “subject” refers to humans or other non-human animals including, e.g., non-human primates, rodents, canines, felines, equines, ovines, bovines, porcines, and the like.

The terms “treat,” “treating,” and “treatment” refer to delaying the onset of, retarding or reversing the progress of, or alleviating or preventing either the disease or condition to which the term applies, or one or more symptoms of such disease or condition.

### BRIEF DESCRIPTION OF THE DRAWINGS

FIG. 1 illustrates a flow chart of the combination of mesenchymal stem cells with a bone substrate;

FIG. 2 illustrates a prior art example of a pellet of a stromal vascular fraction containing the desired stem cells and unwantedcells;

FIGS. 3A-3D illustrates various examples of strips (FIGS. 3A and 3B) and dowels (FIGS. 3C and 3D) which have a 3-D cancellous matrix structure and mesenchymal stem cells (MSCs) may adhere to;

FIG. 4 illustrates a standard curve of total live ASCs using the CCK-8 assay;

FIGS. 5A-5F illustrates mineral deposition by ASCs cultured in osteogenic medium; and

FIG. 6 illustrates H&E staining showed that cells adhered to the bone surface.

FIG. 7 illustrates a flow chart of an exemplary method of combining mesenchymal stem cells with an osteochondral allograft;

FIG. 8 illustrates a flow chart of an exemplary method of combining mesenchymal stem cells with decellularized, morselized cartilage;

FIG. 9 illustrates an exemplary osteochondral allograft;

FIG. 10 illustrates H&E staining of a cartilage control sample; and

FIG. 11 illustrates H&E staining of adiposed-derived stem cells seeded cartilage.

FIGS. 12A-12C show visual assessment of “Original”, “Rinsed”, and “Final” wells, respectively, as viewed under inverted microscope, representative of either Group A (DPBS stored samples, rinsed in DPBS/1% PSA) and Group B (DPBS stored samples, rinsed in DMEM-F12/20% FBS/1% PSA) samples.

FIGS. 13A and 13B show wells containing Group C original epidermal facing surface or basement membrane (“Top”) and Group C original deeper derma or hypodermal facing surface (“Bottom”) samples having some live cells adhered to the plates.

FIG. 14 shows control wells (cells only) containing elongated, healthy looking cells near confluence.

FIGS. 15A-15B show recoverable cell populations from seeded samples. FIG. 15A shows Group C (media stored samples, rinsed in DMEM-F12/20% FBS/1% PSA) “Top” seeded cells, released. FIG. 15B shows unseeded control, no cells released from skin.

FIG. 16 shows comparison of average number of total and live cells, and number of cells positive for various CD markers, between the lipoaspirate, meat grinder+rinse, and meat grinder no rinse methods of isolating a stromal vascular fraction from adipose tissue.

### DETAILED DESCRIPTION

### I. Introduction

Provided are stem cell seeded products and methods relating thereto. The stem cells are mesenchymal stem cells obtained from various donor tissues. In some instances, the tissues may be adipose tissue, muscle tissue, or bone marrow tissue. The mesenchymal stem cells are seeded onto tissue-based substrates. The substrate may be a bone material or non-bone material. The substrate may be a collagen-based material. In some instances, the non-bone material may be cartilage or soft tissue. Mesenchymal stem cells are seeded directly on the substrate after isolation, for example, without culturing or in vitro expansion. Mesenchymal stem cells may be seeded on the substrate as part of a heterogeneous cell population containing mesenchymal stem cells and unwanted cells.

The tissue based substrates may be derived from a variety of tissues. For example, bone substrates may be cortical bone, cancellous bone, or a combination thereof. Substrates may also include cartilage tissue or osteochondral tissue comprising bone and cartilage. Collagen matrices may be derived from any collagenous tissue, including soft tissue. In some instances, a collagen matrix substrate may not be derived from articular cartilage or bone. In some instances, a collagen matrix may be engineered from one or more purified types of collagen.

In some instances, the substrate may be processed to be acellular or partially decellularized. For example, a bone substrate may be decellularized. Such bone substrates may be partially or fully demineralized. In another example, a cartilage substrate may be fully or partially decellularized. In another example, a collagen matrix may be fully or partially decellularized.

In some instances, the substrate may be processed into particulate form. For example, a bone substrate may be ground bone. In another example, a cartilage substrate may be morselized cartilage. Collagen matrices may also be in particulate form.

The cell suspension may be derived from a variety of tissues. Such tissues include adipose tissue, muscle tissue, birth tissue (such as amnion or amniotic fluid), skin tissue, bone tissue, or bone marrow tissue. The tissue is processed to generate a cell suspension containing mesenchymal stem cells and unwanted cells that are non-adherent (anchorage-independent). This processing can include enzymatic digestion of the tissue to release the cells from the other tissue components. In some instances, the digested tissue can be centrifuged to separate the cells from other tissue components. In some instances, tissue may be centrifuged without prior digestion (e.g., bone marrow tissue).

While in vitro culturing of heterogeneous cell suspensions containing mesenchymal stem cells is known to enrich for the mesenchymal stem cells, the cell suspensions described herein are not cultured in vitro prior to seeding on the tissue-based substrate. Rather, the cell suspensions derived from the donor tissue are seeded on the tissue-based substrate without prior in vitro culturing. The seeded substrate is then incubated for a sufficient time to allow the mesenchymal stem cells to adhere to the substrate, thereby forming a seeded substrate. Once the cells have adhered, the seeded substrate is rinsed to remove unwanted cells, thereby providing the stem cell seeded product of the disclosure. The seeded substrates are not cultured to proliferate or differentiate the seeded cells on the substrate. In some instances, the product may be placed in a cryopreservation media.

### II. Bone Constructs

A. Introduction

Unless otherwise described, human adult stem cells are generally referred to as mesenchymal stem cells or MSCs. MSCs are pluripotent cells that have the capacity to differentiate in accordance with at least two discrete development pathways. Adipose-derived stem cells or ASCs are stem cells that are derived from adipose tissue. Stromal Vascular Fraction or SVF generally refers to the centrifuged cell pellet obtained after digestion of tissue containing MSCs, though other methods of obtaining SVF may be used. In one embodiment, the pellet may include multiple types of cells, including stem cells (e.g., one or more of hematopoietic stem cells, epithelial progenitor cells, and mesenchymal stem cells). In an embodiment, mesenchymal stem cells are filtered from other cells by their adherence to a bone substrate, while the other cells (i.e., unwanted cells) do not adhere to the bone substrate. Cells that do not adhere to the bone substrate are unwanted cells.

Adipose derived stem cells may be isolated from cadavers and characterized using flow cytometry and tri-lineage differentiation (osteogenesis, chondrogenesis and adipogenesis) in vitro. The final product may be characterized using histology for microstructure and biochemical assays for cell count. This consistent cell-based product may be useful for bone regeneration.

Tissue engineering and regenerative medicine approaches offer great promise to regenerate bodily tissues. The most widely studied tissue engineering approaches, which are based on seeding and in vitro culturing of cells within scaffolds before implantation, focus on the cell source and the ability to control cell proliferation and differentiation. Many researchers have demonstrated that adipose tissue-derived stem cells (ASCs) possess multiple differentiation capacities. See, for example, the following, which are incorporated by reference:
Rada, T., R. L. Reis, and M. E. Gomes, Adipose Tissue-Derived Stem Cells and Their Application in Bone and Cartilage Tissue Engineering. Tissue Eng Part B Rev, 2009.
Ahn, H. H., et al., In vivo osteogenic differentiation of human adipose-derived stem cells in an injectable in situ forming gel scaffold. Tissue Eng Part A, 2009. 15(7): p. 1821-32.
Anghileri, E., et al., Neuronal differentiation potential of human adipose-derived mesenchymal stem cells. Stem Cells Dev, 2008. 17(5): p. 909-16.
Arnalich-Montiel, F., et al., Adipose-derived stem cells are a source for cell therapy of the corneal stroma. Stem Cells, 2008. 26(2): p. 570-9.
Bunnell, B. A., et al., Adipose-derived stem cells: isolation, expansion and differentiation. Methods, 2008. 45(2): p. 115-20.
Chen, R. B., et al., [Differentiation of rat adipose-derived stem cells into smooth-muscle-like cells in vitro]. Zhonghua Nan Ke Xue, 2009. 15(5): p. 425-30.
Cheng, N. C., et al., Chondrogenic differentiation of adipose-derived adult stem cells by a porous scaffold derived from native articular cartilage extracellular matrix. Tissue Eng Part A, 2009. 15(2): p. 231-41.
Cui, L., et al., Repair of cranial bone defects with adipose derived stem cells and coral scaffold in a canine model. Biomaterials, 2007. 28(36): p. 5477-86.
de Girolamo, L., et al., Osteogenic differentiation of human adipose-derived stem cells: comparison of two different inductive media. J Tissue Eng Regen Med, 2007. 1(2): p. 154-7.
Elabd, C., et al., Human adipose tissue-derived multipotent stem cells differentiate in vitro and in vivo into osteocyte-like cells. Biochem Biophys Res Commun, 2007. 361(2): p. 342-8.
Flynn, L., et al., Adipose tissue engineering with naturally derived scaffolds and adipose-derived stem cells. Biomaterials, 2007. 28(26): p. 3834-42.
Flynn, L. E., et al., Proliferation and differentiation of adipose-derived stem cells on naturally derived scaffolds. Biomaterials, 2008. 29(12): p. 1862-71.
Fraser, J. K., et al., Adipose-derived stem cells. Methods Mol Biol, 2008. 449: p. 59-67.
Gimble, J. and F. Guilak, Adipose-derived adult stem cells: isolation, characterization, and differentiation potential. Cytotherapy, 2003. 5(5): p. 362-9.
Gimble, J. M. and F. Guilak, Differentiation potential of adipose derived adult stem (ADAS) cells. Curr Top Dev Biol, 2003. 58: p. 137-60.
Jin, X. B., et al., Tissue engineered cartilage from hTGF beta2 transduced human adipose derived stem cells seeded in PLGA/alginate compound in vitro and in vivo. J Biomed Mater Res A, 2008. 86(4): p. 1077-87.
Kakudo, N., et al., Bone tissue engineering using human adipose-derived stem cells and honeycomb collagen scaffold. J Biomed Mater Res A, 2008. 84(1): p. 191-7.
Kim, H. J. and G. I. Im, Chondrogenic differentiation of adipose tissue-derived mesenchymal stem cells: greater doses of growth factor are necessary. J Orthop Res, 2009. 27(5): p. 612-9.
Kingham, P. J., et al., Adipose-derived stem cells differentiate into a Schwann cell phenotype and promote neurite outgrowth in vitro. Exp Neural, 2007. 207(2): p. 267-74.
Mehlhorn, A. T., et al., Chondrogenesis of adipose-derived adult stem cells in a poly-lactide-co-glycolide scaffold. Tissue Eng Part A, 2009. 15(5): p. 1159-67.
Merceron, C., et al., Adipose-derived mesenchymal stem cells and biomaterials for cartilage tissue engineering. Joint Bone Spine, 2008. 75(6): p. 672-4.
Mischen, B. T., et al., Metabolic and functional characterization of human adipose-derived stem cells in tissue engineering. Plast Reconstr Surg, 2008. 122(3): p. 725-38.
Mizuno, H., Adipose-derived stem cells for tissue repair and regeneration: ten years of research and a literature review. J Nippon Med Sch, 2009. 76(2): p. 56-66.
Tapp, H., et al., Adipose-Derived Stem Cells: Characterization and Current Application in Orthopaedic Tissue Repair. Exp Biol Med (Maywood), 2008.
Tapp, H., et al., Adipose-derived stem cells: characterization and current application in orthopaedic tissue repair. Exp Biol Med (Maywood), 2009. 234(1): p. 1-9.
van Dijk, A., et al., Differentiation of human adipose-derived stem cells towards cardiomyocytes is facilitated by laminin. Cell Tissue Res, 2008. 334(3): p. 457-67.
Wei, Y., et al., A novel injectable scaffold for cartilage tissue engineering using adipose-derived adult stem cells. J Orthop Res, 2008. 26(1): p. 27-33.
Wei, Y., et al., Adipose-derived stem cells and chondrogenesis. Cytotherapy, 2007. 9(8): p. 712-6.
Zhang, Y. S., et al., [Adipose tissue engineering with human adipose-derived stem cells and fibrin glue injectable scaffold]. Zhonghua Yi Xue Za Zhi, 2008. 88(38): p. 2705-9.

Additionally, adipose tissue is probably the most abundant and accessible source of adult stem cells. Adipose tissue derived stem cells have great potential for tissue regeneration. Nevertheless, ASCs and bone marrow-derived stem cells (BMSCs) are remarkably similar with respect to growth and morphology, displaying fibroblastic characteristics, with abundant endoplasmic reticulum and large nucleus relative to the cytoplasmic volume. See, for example, the following, which are incorporated by reference:

Gimble, J. and F. Guilak, Adipose-derived adult stem cells: isolation, characterization, and differentiation potential. Cytotherapy, 2003. 5(5): p. 362-9.
Gimble, J. M. and F. Guilak, Differentiation potential of adipose derived adult stem (ADAS) cells. Curr Top Dev Bioi, 2003. 58: p. 137-60.
Strem, B. M., et al., Multipotential differentiation of adipose tissue-derived stem cells. Keio J Med, 2005. 54(3): p. 132-41.
De Ugarte, D. A., et al., Comparison of multi-lineage cells from human adipose tissue and bone marrow. Cells Tissues Organs, 2003. 174(3): p. 101-9.
Hayashi, O., et al., Comparison of osteogenic ability of rat mesenchymal stem cells from bone marrow, periosteum, and adipose tissue. Calcif Tissue Int. 2008. 82(3): p. 238-47.
Kim, Y., et al., Direct comparison of human mesenchymal stem cells derived from adipose tissues and bone marrow in mediating neovascularization in response to vascular ischemia. Cell Physiol Biochem, 2007. 20(6): p. 867-76.
Lin, L., et al., Comparison of osteogenic potentials of BMP4 transduced stem cells from autologous bone marrow and fat tissue in a rabbit model of calvarial defects. Calcif Tissue Int, 2009. 85(1): p. 55-65.
Niemeyer, P., et al., Comparison of immunological properties of bone marrow stromal cells and adipose tissue-derived stem cells before and after osteogenic differentiation in vitro. Tissue Eng, 2007. 13(1): p. 111-21.
Noel, D., et al., Cell specific differences between human adipose-derived and mesenchymal-stromal cells despite similar differentiation potentials. Exp Cell Res, 2008. 314(7): p. 1575-84.
Yoo, K. H., et al., Comparison of immunomodulatory properties of mesenchymal stem cells derived from adult human tissues. Cell Immunol, 2009.
Yoshimura, H., et al., Comparison of rat mesenchymal stem cells derived from bone marrow, synovium, periosteum, adipose tissue, and muscle. Cell Tissue Res, 2007. 327(3): p. 449-62.

Other common characteristics of ASCs and BMSCs can be found in the transcriptional and cell surface profile. Several studies have already been done in the field of bone tissue engineering using ASCs. See, for example, the following, which are incorporated by reference:
Rada, T., R. L. Reis, and M. E. Gomes, Adipose Tissue-Derived Stem Cells and Their Application in Bone and Cartilage Tissue Engineering. Tissue Eng Part B Rev, 2009.
Tapp, H., et al., Adipose-Derived Stem Cells: Characterization and Current Application in Orthopaedic Tissue Repair. Exp Biol Med (Maywood), 2008.
Tapp, H., et al., Adipose-derived stem cells: characterization and current application in orthopaedic tissue repair. Exp Biol Med (Maywood), 2009. 234(1): p. 1-9.
De Girolamo, L., et al., Human adipose-derived stem cells as future tools in tissue regeneration: osteogenic differentiation and cell-scaffold interaction. Int J Artif Organs, 2008. 31(6): p. 467-79.
Di Bella, C., P. Farlie, and A. J. Penington, Bone regeneration in a rabbit critical-sized skull defect using autologous adipose-derived cells. Tissue Eng Part A, 2008. 14(4): p. 483-90.
Grewal, N. S., et al., BMP-2 does not influence the osteogenic fate of human adipose-derived stem cells. Plast Reconstr Surg, 2009. 123(2 Suppl): p. 158S-65S.
Li, H., et al., Bone regeneration by implantation of adipose-derived stromal cells expressing BMP-2. Biochem Biophys Res Commun, 2007. 356(4): p. 836-42.
Yoon, E., et al., In vivo osteogenic potential of human adipose-derived stem cells/poly lactide-co-glycolic acid constructs for bone regeneration in a rat critical-sized calvarial defect model. Tissue Eng, 2007. 13(3): p. 619-27.

These studies have demonstrated that stem cells obtained from adipose tissue exhibit good attachment properties to most of the material surfaces in vitro and the capacity to differentiate into osteoblastic-like cells in vitro and in vivo. Recently it has been shown that ASCs may stimulate the vascularization process. See, for example, the following, which are incorporated by reference:
Butt, O. I., et al., Stimulation of peri-implant vascularization with bone marrow-derived progenitor cells: monitoring by in vivo EPR oximetry. Tissue Eng, 2007. 13(8): p. 2053-61.
Rigotti, G., et al., Clinical treatment of radiotherapy tissue damage by lipoaspirate transplant: a healing process mediated by adipose-derived adult stem cells. Plast Reconstr Surg, 2007. 119(5): p. 1409-22; discussion 1423-4.

Demineralized bone substrate, as an allogeneic material, is a promising bone tissue-engineering scaffold due to its close relation to autologous bone in terms of structure and function. Combined with MSCs, these scaffolds have been demonstrated to accelerate and enhance bone formation within osseous defects when compared with the matrix alone. See, for example, the following, which are incorporated by reference:
Chen, L. Q., et al., [Study of MSCs in vitro cultured on demineralized bone matrix of mongrel]. Shanghai Kou Qiang Yi Xue, 2007. 16(3): p. 255-8.
Gamradt, S. C. and J. R. Lieberman, Bone graft for revision hip arthroplasty: biology and future applications. Clin Orthop Relat Res, 2003(417): p. 183-94.
Honsawek, S., D. Dhitiseith, and V. Phupong, Effects of demineralized bone matrix on proliferation and osteogenic differentiation of mesenchymal stem cells from human umbilical cord. J Med Assoc Thai, 2006. 89 Suppl 3: p. S189-95.
Kasten, P., et al., [Induction of bone tissue on different matrices: an in vitro and a in vivo pilot study in the SCID mouse]. Z Orthop Ihre Grenzgeb, 2004.142(4): p. 467-75.
Kasten, P., et al., Ectopic bone formation associated with mesenchymal stem cells in a resorbable calcium deficient hydroxyapatite carrier. Biomaterials, 2005. 26(29): p. 5879-89.
Qian, Y., Z. Shen, and Z. Zhang, [Reconstruction of bone using tissue engineering and nanoscale technology]. Zhongguo Xiu Fu Chong Jian Wai Ke Za Zhi, 2006. 20(5): p. 560-4.
Reddi, A. H., Role of morphogenetic proteins in skeletal tissue engineering and regeneration. Nat Biotechnol, 1998. 16(3): p. 247-52.
Reddi, A. H., Morphogenesis and tissue engineering of bone and cartilage: inductive signals, stem cells, and biomimetic biomaterials. Tissue Eng, 2000.6(4): p. 351-9.
Tsiridis, E., et al., In vitro and in vivo optimization of impaction allografting by demineralization and addition of rh-OP-1. J Orthop Res, 2007. 25(11): p. 1425-37.
Xie, H., et al., The performance of a bone-derived scaffold material in the repair of critical bone defects in a rhesus monkey model. Biomaterials, 2007.28(22): p. 3314-24.
Liu, G., et al., Tissue-engineered bone formation with cryopreserved human bone marrow mesenchymal stem cells. Cryobiology, 2008. 56(3): p. 209-15.
Liu, G., et al., Evaluation of partially demineralized osteoporotic cancellous bone matrix combined with human bone marrow stromal cells for tissue engineering: an in vitro and in vivo study. Calcif Tissue Int, 2008. 83(3): p. 176-85.
Liu, G., et al., Evaluation of the viability and osteogenic differentiation of cryopreserved human adipose-derived stem cells. Cryobiology, 2008. 57(1): p. 18-24.

B. Compositions and Methods

As discussed herein, bone substrates seeded with stem cell containing cell populations may be characterized in terms of microstructure, cell number and cell identity using histology, biochemical assays, and flow cytometry. In an embodiment, these substrates may include bone material which has been previously subjected to a demineralization process.

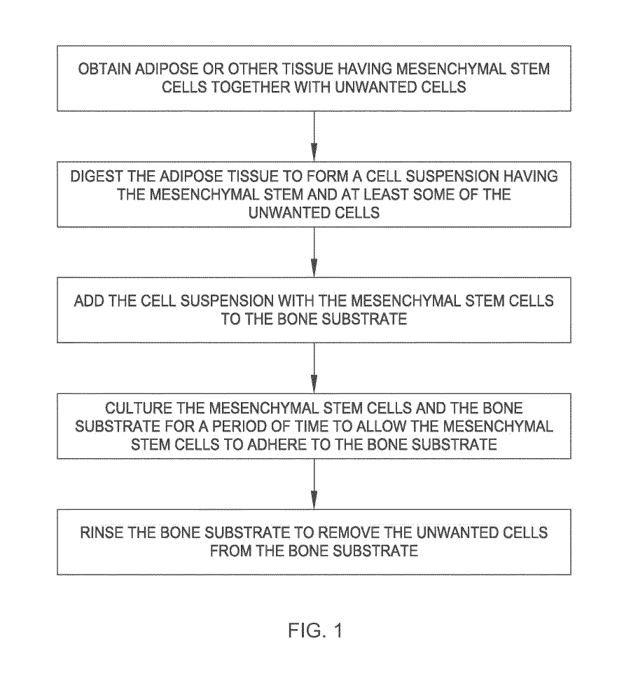

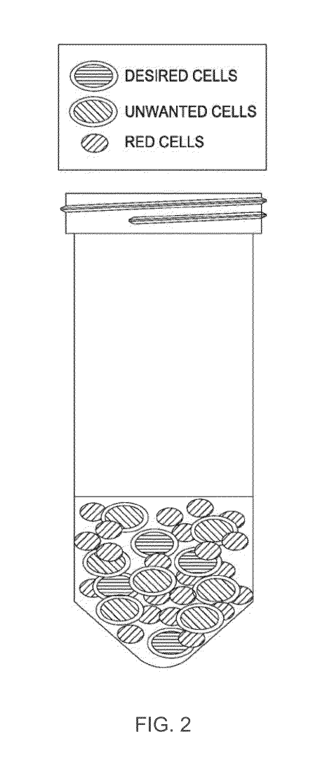

FIG. 1 is a flow chart of a process for making an allograft with stem cells product. In an embodiment, a stromal vascular fraction may be used to seed the allograft. It should be apparent from the present disclosure that the term “seed” relates to addition and placement of the stem cells within, or at least in attachment to, the allograft, but is not limited to a specific process. FIG. 2 illustrates a pellet of the stromal vascular fraction containing the desired stem cells.

In an exemplary embodiment, a method of combining mesenchymal stem cells with a bone substrate is provided. The method may include obtaining adipose tissue having the mesenchymal stem cells together with unwanted cells. Unwanted cells may include hematopoietic stem cells and other stromal cells. The method may further include processing, such as by digesting, the adipose tissue to form a cell suspension having the mesenchymal stem cells and at least some or all of the unwanted cells. In another embodiment, this may be followed by negatively depleting some of the unwanted cells and other constituents to concentrate mesenchymal stem cells.

Next, the method includes adding the cell suspension with the mesenchymal stem cells to the bone substrate. This may be followed by culturing the mesenchymal stem cells and the bone substrate for a period of time to allow the mesenchymal stem cells to adhere to the bone substrate. In order to provide a desired product, the method includes rinsing the bone substrate to remove the unwanted cells from the bone substrate.

In one embodiment, an allograft product may include a combination of mesenchymal stem cells with a bone substrate such that the combination is manufactured by the above exemplary embodiment.

In an embodiment, the adipose tissue may be obtained from a cadaveric donor. A typical donor yields 2 liters of adipose containing 18 million MSCs. In one embodiment, a bone substrate may be from the same cadaveric donor as the adipose tissue. In another embodiment, the adipose tissue may be obtained from a patient. In addition, both the bone substrate and the adipose tissue may be obtained from the same patient. This may include, but is not limited to, removal of a portion of the ilium (e.g., the iliac crest) from the donor by a surgical procedure and adipose cells may be removed using liposuction. Other sources, and combination of sources, of adipose tissue, other tissues, and bone substrates may be utilized.

Optionally, the adipose tissue may be washed prior to or during processing (e.g., digestion). Washing may include using a thermal shaker at 75 RPM at 37° C. for at least 10 minutes. Washing the adipose tissue may include washing with a volume of PBS substantially equal to the adipose tissue. In an embodiment, washing the adipose tissue includes washing with the PBS with 1% penicillin and streptomycin at about 37° C.

For example, washing the adipose tissue may include agitating the tissue and allowing phase separation for about 3 to 5 minutes. This may be followed by aspirating off a infranatant solution. The washing may include repeating washing the adipose tissue multiple times until a clear infranatant solution is obtained. In one embodiment, washing the adipose tissue may include washing with a volume of growth media substantially equal to the adipose tissue.

In another exemplary embodiment, a method of combining mesenchymal stem cells with a bone substrate is provided. The method may include obtaining bone marrow tissue having the mesenchymal stem cells together with unwanted cells. Unwanted cells may include hematopoietic stem cells and other stromal cells. The method may further include processing (e.g., digesting) the bone marrow tissue to form a cell suspension having the mesenchymal stem cells and the unwanted cells. In another embodiment, this may be followed by naturally selecting MSCs and depleting some of the unwanted cells and other constituents to concentrate mesenchymal stem cells.

Next, the method includes adding the cell suspension with the mesenchymal stem cells to the bone substrate. This may be followed by culturing the mesenchymal stem cells and the bone substrate for a period of time to allow the mesenchymal stem cells to adhere to the bone substrate. In order to provide a desired product, the method includes rinsing the bone substrate to remove the unwanted cells from the bone substrate.

In one embodiment, an allograft product may include a combination of mesenchymal stem cells with a bone substrate such that the combination is manufactured by the above exemplary embodiment.

In another exemplary embodiment, a method of combining mesenchymal stem cells with a bone substrate is provided. The method may include obtaining muscle tissue having the mesenchymal stem cells together with unwanted cells. Unwanted cells may include hematopoietic stem cells and other stromal cells. The method may further include processing (e.g., digesting) the muscle tissue to form a cell suspension having the mesenchymal stem cells and the unwanted cells. In another embodiment, this may be followed by naturally selecting MSCs to concentrate mesenchymal stem cells.

Next, the method includes adding the cell suspension with the mesenchymal stem cells to the bone substrate. This may be followed by culturing the mesenchymal stem cells and the bone substrate for a period of time to allow the mesenchymal stem cells to adhere to the bone substrate. In order to provide a desired product, the method includes rinsing the bone substrate to remove the unwanted cells from the bone substrate.

In one embodiment, an allograft product may include combination of mesenchymal stem cells with a bone substrate such that the combination is manufactured by the above exemplary embodiment.

In another exemplary embodiment, a method of combining mesenchymal stem cells with a bone substrate is provided. The method may include obtaining tissue having the mesenchymal stem cells together with unwanted cells. Unwanted cells may include hematopoietic stem cells and other stromal cells.

The method may further include processing (e.g., digesting) the tissue to form a cell suspension having the mesenchymal stem cells and at least some of the unwanted cells. In another embodiment, this may be followed by negatively depleting some of the unwanted cells and other constituents to concentrate mesenchymal stem cells.

Next, the method includes adding the cell suspension with the mesenchymal stem cells to the bone substrate. In an embodiment, this substrate may include a bone material which has been subjected to a demineralization process. In another embodiment, this substrate may be a non-bone material, which may include (but is not limited to) a collagen based material. This may be followed by culturing the mesenchymal stem cells and the bone substrate for a period of time to allow the mesenchymal stem cells to adhere to the bone substrate. In order to provide a desired product, the method includes rinsing the bone substrate to remove the unwanted cells from the bone substrate.

In one embodiment, an allograft product may include a combination of mesenchymal stem cells with a bone substrate such that the combination is manufactured by the above exemplary embodiment.

Digesting the cell suspension may include making a collagenase I solution, and filtering the solution through a 0.2 μm filter unit, mixing the adipose tissue with the collagenase I solution, and adding the cell suspension mixed with the collagenase I solution to a shaker flask. Digesting the cell suspension may further include placing the shaker with continuous agitation at about 75 RPM for about 45 to 60 minutes so as to provide the adipose tissue with a visually smooth appearance.

Digesting the cell suspension may further include aspirating supernatant containing mature adipocytes so as to provide a pellet, which may be referred to as a stromal vascular fraction. (See, for example, FIG. 2.) Prior to seeding, a lab sponge or other mechanism may be used to pat dry bone substrate.

In one embodiment, adding the cell suspension with the mesenchymal stem cells to the bone substrate may include using a cell pellet for seeding onto the bone substrate. In an embodiment, adding the cell suspension with the mesenchymal stem cells to the bone substrate may include using a cell pellet for seeding onto the bone substrate. In another embodiment, adding the cell suspension with the mesenchymal stem cells to the bone substrate may include using a cell pellet for seeding onto the bone substrate of cortical bone. In another embodiment, adding the cell suspension with the mesenchymal stem cells to the bone substrate may include adding the cell pellet onto the bone substrate of cancellous bone. In another embodiment, adding the cell suspension with the mesenchymal stem cells to the bone substrate may include adding the cell pellet onto the bone substrate of ground bone. In another embodiment, adding the cell suspension with the mesenchymal stem cells to the bone substrate may include adding the cell pellet onto the bone substrate of cortical/cancellous bone. In another embodiment, adding the cell suspension with the mesenchymal stem cells to the bone substrate may include adding the cell pellet onto the bone substrate of demineralized cancellous bone.

In an embodiment, the method may include placing the bone substrate into a cryopreservation media after rinsing the bone substrate. This cryopreservation media may be provided to store the final products. For example, the method may include maintaining the bone substrate into a frozen state after rinsing the bone substrate to store the final products. The frozen state may be at about negative 80° C.

In another embodiment, Ficoll density solution may be utilized. For example, negatively depleting the concentration of the mesenchymal stem cells may include adding a volume of PBS and a volume of Ficoll density solution to the adipose solution. The volume of PBS may be 5 ml and the volume of Ficoll density solution may be 25 ml with a density of 1.073 g/ml. Negatively depleting the concentration of the mesenchymal stem cells may also include centrifuging the adipose solution at about 1160 g for about 30 minutes at about room temperature. In one embodiment, the method may include stopping the centrifuging the adipose solution without using a brake.

Negatively depleting the concentration of the mesenchymal stem cells is optional and may next include collecting an upper layer and an interface containing nucleated cells, and discarding a lower layer of red cells and cell debris. Negatively depleting the concentration of the mesenchymal stem cells may also include adding a volume of D-PBS of about twice an amount of the upper layer of nucleated cells, and inverting a container containing the cells to wash the collected cells. Negatively depleting the concentration of the mesenchymal stem cells may include centrifuging the collected cells to pellet the collected cells using the break during deceleration.

In an embodiment, negatively depleting the concentration of the mesenchymal stem cells may further include centrifuging the collected cells at about 900 g for about 5 minutes at about room temperature. Negatively depleting some of the unwanted cells may include discarding a supernatant after centrifuging the collected cells, and resuspending the collected cells in a growth medium.

In one embodiment, adding the cell suspension with the mesenchymal stem cells to the bone substrate may include adding the cell pellet onto the bone substrate. Adding the solution with the mesenchymal stem cells to the bone substrate may include adding cell pellet onto the bone substrate which was subjected to a demineralization process. In another embodiment, adding the cell suspension with the mesenchymal stem cells to the bone substrate may include adding the cell pellet onto the bone substrate of cortical bone. In an embodiment, adding the cell suspension with the mesenchymal stem cells to the bone substrate includes adding the cell pellet onto the bone substrate of cancellous bone. In another embodiment, adding the cell suspension with the mesenchymal stem cells to the bone substrate may include adding the cell pellet onto the bone substrate of ground bone. In another embodiment, adding the cell suspension with the mesenchymal stem cells to the bone substrate may include adding the cell pellet onto the bone substrate of cortical cancellous bone. In another embodiment, adding the cell suspension with the mesenchymal stem cells to the bone substrate may include adding the cell pellet onto the bone substrate of demineralized cancellous bone.

In an embodiment, the method may further include placing the bone substrate into a cryopreservation media after rinsing the bone substrate. This cryopreservation media may be provided to store the final products. The method may include maintaining the bone substrate into a frozen state after rinsing the bone substrate to store the final products. The frozen state may be at about negative 80° C.

The seeded allografts are cultured for a period of time to allow the mesenchymal stem cells to adhere to the bone substrate. The unwanted cells were rinsed and removed from the bone substrate. After culturing, a lab sponge or other mechanism may be used to pat dry the bone substrate.

The mesenchymal stem cells are anchorage dependent. The mesenchymal stem cells naturally adhere to the bone substrate. The mesenchymal stem cells are non-immunogenic and regenerate bone. The unwanted cells are generally anchorage independent. This means that the unwanted cells generally do not adhere to the bone substrate. The unwanted cells may be immunogenic and may create blood and immune system cells. For cell purification during a rinse, mesenchymal stem cells adhere to the bone while unwanted cells, such as hematopoietic stem cells, are rinsed away leaving a substantially uniform population of mesenchymal stem cells on the bone substrate.

The ability to mineralize the extracellular matrix and to generate bone is not unique to MSCs. In fact, ASCs possess a similar ability to differentiate into osteoblasts under similar conditions. Human ASCs offer a unique advantage in contrast to other cell sources. The multipotent characteristics of ASCs, as wells as their abundance in the human body, make these cells a desirable source in tissue engineering applications.

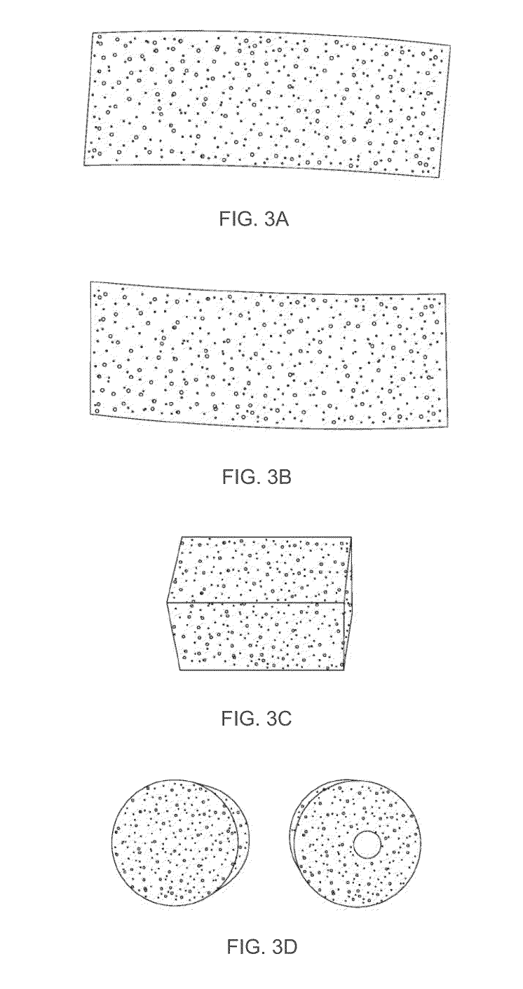

In various embodiments, bone substrates (e.g., cortical cancellous dowels, strips, cubes, blocks, discs, and granules, as well as other substrates formed in dowels, strips, cubes, blocks, discs, and granules) may be subjected to a demineralization process to remove blood, lipids and other cells so as to leave a matrix. FIGS. 3A-3D illustrates various examples of strips (FIGS. 3A and 3B) and dowels (FIGS. 3C and 3D). Generally, these substrates may have a 3-D cancellous matrix structure, which MSCs may adhere to.

In addition, this method and combination product involve processing that does not alter the relevant biological characteristics of the tissue. Processing of the adipose/stem cells may involve the use of antibiotics, cell media, collagenase. None of these affects the relevant biological characteristics of the stem cells. The relevant biological characteristics of these mesenchymal stem cells are centered on renewal and repair. The processing of the stem cells does not alter the cell's ability to continue to differentiate and repair.

In the absence of stimulation or environmental cues, mesenchymal stem cells (MSCs) remain undifferentiated and maintain their potential to form tissue such as bone, cartilage, fat, and muscle. Upon attachment to an osteoconductive matrix, MSCs have been shown to differentiate along the osteoblastic lineage in vivo. See, for example, the following, which are incorporated by reference:
Arinzeh T L, Peter S J, Archambault M P, van den Bas C, Gordon S, Kraus K, Smith A, Kadiyala S. Allogeneic mesenchymal stem cells regenerate bone in a critical sized canine segmental defect. J Bone Joint Surg Am. 2003; 85-A:1927-35.
Bruder S P, Kurth A A, Shea M, Hayes W C, Jaiswal N, Kadiyala S. Bone regeneration by implantation of purified, culture-expanded human mesenchymal stem cells, J Orthop Res. 1998; 16:155-62.

C. Exemplary Features

In one instance, there is provided a method of combining mesenchymal stem cells with a bone substrate, the method comprising obtaining adipose tissue having the mesenchymal stem cells together with unwanted cells; processing (e.g., digesting) the adipose tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In some instances, the obtaining the adipose tissue includes recovery from a cadaveric donor. In some cases, the bone substrate is from a cadaveric donor, and the obtaining the adipose tissue includes recovery from the same cadaveric donor as the bone substrate. In some instances, the obtaining the adipose tissue includes recovery from a patient. In some cases, the bone substrate is from a cadaveric donor, and the obtaining the adipose tissue includes recovery from the same patient as the bone substrate. In some instances, the digesting the adipose tissue includes making a collagenase I solution, and filtering the solution through a 0.2 μm filter unit, mixing the adipose with the collagenase I solution, and adding the adipose with the collagenase I solution to a shaker flask. In some cases, the digesting the adipose further includes placing the shaker with continuous agitation at about 75 RPM for about 45 to 60 minutes so as to provide the adipose tissue with a visually smooth appearance. In some instances, the digesting the adipose further includes aspirating a supernatant containing mature adipocytes so as to provide a pellet. In some cases, the adding the suspension with the mesenchymal stem cells to the bone substrate includes adding the cell suspension onto the bone substrate. In some instances, the adding the suspension with the mesenchymal stem cells to the bone substrate includes adding the cell suspension onto a bone substrate previously subjected to a demineralization process. In some cases, the adding the suspension with the mesenchymal stem cells to the bone substrate includes adding the cell suspension onto cortical bone. In some instances, the adding the suspension with the mesenchymal stem cells to the bone substrate includes adding the cell suspension onto cancellous bone. In some cases, the adding the suspension with the mesenchymal stem cells to the bone substrate includes adding the cell suspension onto ground bone. In some instances, the adding the suspension with the mesenchymal stem cells to the bone substrate includes adding the cell suspension onto cortical/cancellous bone. In some cases, the adding the suspension with the mesenchymal stem cells to the bone substrate includes adding the cell suspension onto demineralized cancellous bone. In some cases, the method further includes placing the bone substrate into a cryopreservation media after rinsing the bone substrate to store the final products. In some instances, the method further includes maintaining the bone substrate into a frozen state after rinsing the bone substrate and, in some cases, the frozen state is at about negative 80° C. In some instances, the bone substrate includes a bone substrate previously subjected to a demineralization process. In some cases, the bone substrate includes cortical bone. In some cases, the bone substrate includes cancellous bone. In some instances, the bone substrate includes ground bone. In some cases, the bone substrate includes cortical and cancellous bone. In some cases, the bone substrate includes demineralized cancellous bone.

In another instance, there is provided an allograft product including a combination of mesenchymal stem cells with a bone substrate, and the combination manufactured by obtaining adipose tissue having the mesenchymal stem cells together with unwanted cells; processing (e.g., digesting) the adipose tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In still another instance, there is provided a method of combining mesenchymal stem cells with a bone substrate, the method comprising obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the bone substrate so as to form a seeded bone substrate; culturing the mesenchymal stem cells on the seeded bone substrate for a period of time to allow the mesenchymal stem cells to adhere to the bone substrate; and rinsing the bone substrate to remove the unwanted cells from the bone substrate.

In yet another instance, there is provided an allograft product including a combination of mesenchymal stem cells with a bone substrate, and the combination manufactured by obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the bone substrate by adding the pellet onto the bone substrate so as to form a seeded bone substrate; culturing the mesenchymal stem cells on the seeded bone substrate for a period of time to allow the mesenchymal stem cells to adhere to the bone substrate; and rinsing the bone substrate to remove the unwanted cells from the bone substrate.

In an instance, there is provided a method of combining mesenchymal stem cells with a bone substrate, the method comprising obtaining tissue having the mesenchymal stem cells together with unwanted cells; processing (e.g., digesting) the tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In some instances, the obtaining the tissue includes recovery from a cadaveric donor. In some cases, the bone substrate is from a cadaveric donor, and the obtaining the tissue includes recovery from the same cadaveric donor as the bone substrate. In some instances, the obtaining the tissue includes recovery from a patient. In some cases, the bone substrate is from a cadaveric donor, and the obtaining the tissue includes recovery from the same patient as the bone substrate. In some instances, the bone substrate includes a bone substrate previously subjected to a demineralization process. In some cases, the bone substrate includes cortical bone. In some cases, the bone substrate includes cancellous bone. In some cases, the bone substrate includes ground bone. In some cases, the bone substrate includes cortical and cancellous bone. In some cases, the bone substrate includes demineralized cancellous bone.

In another instance, there is provided an allograft product including a combination of mesenchymal stem cells with a bone substrate, and the combination manufactured by obtaining tissue having the mesenchymal stem cells together with unwanted cells; processing (e.g., digesting) the tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In still another instance, there is provided a method of combining mesenchymal stem cells with a bone substrate, the method comprising obtaining bone marrow tissue having the mesenchymal stem cells together with unwanted cells; processing (e.g., digesting) the bone marrow tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In some instances, the obtaining the bone marrow tissue includes recovery from a cadaveric donor. In some cases, the bone substrate is from a cadaveric donor, and the obtaining the bone marrow tissue includes recovery from the same cadaveric donor as the bone substrate. In some instances, the obtaining the bone marrow tissue includes recovery from a patient. In some cases, the bone substrate is from a cadaveric donor, and the obtaining the bone marrow tissue includes recovery from the same patient as the bone substrate. In some instances, the bone substrate includes a bone substrate previously subjected to a demineralization process. In some cases, the bone substrate includes cortical bone. In some cases, the bone substrate includes cancellous bone. In some cases, the bone substrate includes ground bone. In some cases, the bone substrate includes cortical and cancellous bone. In some cases, the bone substrate includes demineralized cancellous bone.

In yet another instance, there is provided an allograft product including a combination of mesenchymal stem cells with a bone substrate, and the combination manufactured by obtaining bone marrow tissue having the mesenchymal stem cells together with unwanted cells; processing (e.g., digesting) the bone marrow tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells and the bone substrate for a period of time to allow the mesenchymal stem cells to adhere to the bone substrate; and rinsing the bone substrate to remove the unwanted cells from the bone substrate.

In an instance, there is provided a method of combining mesenchymal stem cells with a bone substrate, the method comprising obtaining muscle tissue having the mesenchymal stem cells together with unwanted cells; processing (e.g., digesting) the muscle tissue to form a cell suspension having the mesenchymal stem cells the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

In some instances, the obtaining the muscle tissue includes recovery from a cadaveric donor. In some cases, the bone substrate is from a cadaveric donor, and the obtaining the muscle tissue includes recovery from the same cadaveric donor as the bone substrate. In some instances, the obtaining the muscle tissue includes recovery from a patient. In some cases, the bone substrate is from a cadaveric donor, and the obtaining the muscle tissue includes recovery from the same patient as the bone substrate. In some instances, the bone substrate includes a bone substrate previously subjected to a demineralization process. In some cases, the bone substrate includes cortical bone. In some cases, the bone substrate includes cancellous bone. In some cases, the bone substrate includes ground bone. In some cases, the bone substrate includes cortical and cancellous bone. In some cases, the bone substrate includes demineralized cancellous bone.

In another instance, there is provided an allograft product including a combination of mesenchymal stem cells with a bone substrate, and the combination manufactured by obtaining muscle tissue having the mesenchymal stem cells together with unwanted cells; processing (e.g., digesting) the muscle tissue to form a cell suspension having the mesenchymal stem cells and the unwanted cells; adding the cell suspension with the mesenchymal stem cells to seed the bone substrate so as to form a seeded bone substrate; culturing (incubating) the mesenchymal stem cells on the seeded bone substrate for a period of time to allow the mesenchymal stem cells to adhere to the bone substrate; and rinsing the bone substrate to remove the unwanted cells from the bone substrate.

### III. Cartilage Constructs

A. Introduction

Unless otherwise described, human adult stem cells are generally referred to as mesenchymal stem cells or MSCs. MSCs are pluripotent cells that have the capacity to differentiate in accordance with at least two discrete development pathways. Adipose-derived stem cells or ASCs are stem cells that are derived from adipose tissue. Stromal Vascular Fraction or SVF generally refers to the centrifuged cell pellet obtained after digestion of tissue containing MSCs. Other methods of obtaining SVF may be used as well. In one embodiment, the SVF pellet may include multiple types of stem cells. These stem cells may include, for example, one or more of hematopoietic stem cells, epithelial stem cells, and mesenchymal stem cells. In an embodiment, mesenchymal stem cells are filtered from other stem cells by their adherence to an osteochondral graft (or cartilage or morselized cartilage), while the other stem cells (i.e., unwanted cells) do not adhere to the osteochondral graft (or cartilage or morselized cartilage). Other cells that do not adhere to the osteochondral graft (or cartilage or morselized cartilage) may also be included in these unwanted cells.

Adipose derived stem cells may be isolated from cadavers and characterized using flow cytometry and tri-lineage differentiation (osteogenesis, chondrogenesis and adipogenesis). The final product may be characterized using histology for microstructure and biochemical assays for cell count. This consistent cell-based product may be useful for osteochondral graft (or cartilage or morselized cartilage) regeneration.

Tissue engineering and regenerative medicine approaches offer great promise to regenerate bodily tissues. The most widely studied tissue engineering approaches, which are based on seeding and in vitro culturing of cells within scaffolds before implantation, focus on the cell source and the ability to control cell proliferation and differentiation. Many researchers have demonstrated that adipose tissue-derived stem cells (ASCs) possess multiple differentiation capacities. See, for example, the following, which are incorporated by reference:
Rada, T., R. L. Reis, and M. E. Gomes, Adipose Tissue-Derived Stem Cells and Their Application in Bone and Cartilage Tissue Engineering. Tissue Eng Part B Rev, 2009.
Ahn, H. H., et al., In vivo osteogenic differentiation of human adipose-derived stem cells in an injectable in situ-forming gel scaffold. Tissue Eng Part A, 2009. 15(7): p. 1821-32.
Anghileri, E., et al., Neuronal differentiation potential of human adipose-derived mesenchymal stem cells. Stem Cells Dev, 2008. 17(5): p. 909-16.
Arnalich-Montiel, F., et al., Adipose-derived stem cells are a source for cell therapy of the corneal stroma. Stem Cells, 2008. 26(2): p. 570-9.
Bunnell, B. A., et al., Adipose-derived stem cells: isolation, expansion and differentiation. Methods, 2008. 45(2): p. 115-20.
Chen, R. B., et al., [Differentiation of rat adipose-derived stem cells into smooth-muscle-like cells in vitro]. Zhonghua Nan Ke Xue, 2009. 15(5): p. 425-30.
Cheng, N. C., et al., Chondrogenic differentiation of adipose-derived adult stem cells by a porous scaffold derived from native articular cartilage extracellular matrix. Tissue Eng Part A, 2009. 15(2): p. 231-41.
Cui, L., et al., Repair of cranial bone defects with adipose derived stem cells and coral scaffold in a canine model. Biomaterials, 2007. 28(36): p. 5477-86.
de Girolamo, L., et al., Osteogenic differentiation of human adipose-derived stem cells: comparison of two different inductive media. J Tissue Eng Regen Med, 2007. 1(2): p. 154-7.
Elabd, C., et al., Human adipose tissue-derived multipotent stem cells differentiate in vitro and in vivo into osteocyte-like cells. Biochem Biophys Res Commun, 2007. 361(2): p. 342-8.
Flynn, L., et al., Adipose tissue engineering with naturally derived scaffolds and adipose-derived stem cells. Biomaterials, 2007. 28(26): p. 3834-42.
Flynn, L. E., et al., Proliferation and differentiation of adipose-derived stem cells on naturally derived scaffolds. Biomaterials, 2008. 29(12): p. 1862-71.
Fraser, J. K., et al., Adipose-derived stem cells. Methods Mol Biol, 2008. 449: p. 59-67.
Gimble, J. and F. Guilak, Adipose-derived adult stem cells: isolation, characterization, and differentiation potential. Cytotherapy, 2003. 5(5): p. 362-9.
Gimble, J. M. and F. Guilak, Differentiation potential of adipose derived adult stem (ADAS) cells. CurrTop Dev Bioi, 2003. 58: p. 137-60.
Jin, X. B., et al., Tissue engineered cartilage from hTGF beta2 transduced human adipose derived stem cells seeded in PLGA/alginate compound in vitro and in vivo. J Biomed Mater Res A, 2008. 86(4): p. 1077-87.
Kakudo, N., et al., Bone tissue engineering using human adipose-derived stem cells and honeycomb collagen scaffold. J Biomed Mater Res A, 2008. 84(1): p. 191-7.
Kim, H. J. and G. I. lm, Chondrogenic differentiation of adipose tissue-derived mesenchymal stem cells: greater doses of growth factor are necessary. J Orthop Res, 2009. 27(5): p. 612-9.
Kingham, P. J., et al., Adipose-derived stem cells differentiate into a Schwann cell phenotype and promote neurite outgrowth in vitro. Exp Neural, 2007. 207(2): p. 267-74.
Mehlhorn, A T., et al., Chondrogenesis of adipose-derived adult stem cells in a poly-lactide-co-glycolide scaffold. Tissue Eng Part A, 2009. 15(5): p. 1159-67.
Merceron, C., et al., Adipose-derived mesenchymal stem cells and biomaterials for cartilage tissue engineering. Joint Bone Spine, 2008. 75(6): p. 672-4.
Mischen, B. T., et al., Metabolic and functional characterization of human adipose-derived stem cells in tissue engineering. Plast Reconstr Surg, 2008. 122(3): p. 725-38.
Mizuno, H., Adipose-derived stem cells for tissue repair and regeneration: ten years of research and a literature review. J Nippon Med Sch, 2009. 76(2): p. 56-66.
Tapp, H., et al., Adipose-Derived Stem Cells: Characterization and Current Application in Orthopaedic Tissue Repair. Exp Bioi Med (Maywood), 2008.
Tapp, H., et al., Adipose-derived stem cells: characterization and current application in orthopaedic tissue repair. Exp Bioi Med (Maywood), 2009. 234(1): p. 1-9.
van Dijk, A., et al., Differentiation of human adipose-derived stem cells towards cardiomyocytes is facilitated by laminin. Cell Tissue Res, 2008. 334(3): p. 457-67.
Wei, Y., et al., A novel injectable scaffold for cartilage tissue engineering using adipose-derived adult stem cells. J Orthop Res, 2008. 26(1): p. 27-33. Wei, Y., et al., Adipose-derived stem cells and chondrogenesis. Cytotherapy, 2007. 9(8): p. 712-6.
Zhang, Y. S., et at., [Adipose tissue engineering with human adipose-derived stem cells and fibrin glue injectable scaffold]. Zhonghua Yi Xue Za Zhi, 2008. 88(38): p. 2705-9.

Additionally, adipose tissue is probably the most abundant and accessible source of adult stem cells. Adipose tissue derived stem cells have great potential for tissue regeneration. Nevertheless, ASCs and bone marrow-derived stem cells (BMSCs) are remarkably similar with respect to growth and morphology, displaying fibroblastic characteristics, with abundant endoplasmic reticulum and large nucleus relative to the cytoplasmic volume. See, for example, the following, which are incorporated by reference:
Gimble, J. and F. Guilak, Adipose-derived adult stem cells: isolation, characterization, and differentiation potential. Cytotherapy, 2003. 5(5): p. 362-9.
Gimble, J. M. and F. Guilak, Differentiation potential of adipose derived adult stem (ADAS) cells. Curr Top Dev Bioi, 2003. 58: p. 137-60.
Strem, B. M., et al., Multipotential differentiation of adipose tissue-derived stem cells. Keio J Med, 2005. 54(3): p. 132-41.
De Ugarte, D. A., et al., Comparison of multi-lineage cells from human adipose tissue and bone marrow. Cells Tissues Organs, 2003. 174(3): p. 101-9.
Hayashi, O., et al., Comparison of osteogenic ability of rat mesenchymal stem cells from bone marrow, periosteum, and adipose tissue. Calcif Tissue Int, 2008. 82(3): p. 238-47.
Kim, Y., et al., Direct comparison of human mesenchymal stem cells derived from adipose tissues and bone marrow in mediating neovascu/arization in response to vascular ischemia. Cell Physiol Biochem, 2007. 20(6): p. 867-76.
Lin, L., et al., Comparison of osteogenic potentials of BMP4 transduced stem cells from autologous bone marrow and fat tissue in a rabbit model of calvarial defects. Calcif Tissue Int, 2009. 85(1): p. 55-65.
Niemeyer, P., et al., Comparison of immunological properties of bone marrow stromal cells and adipose tissue-derived stem cells before and after osteogenic differentiation in vitro. Tissue Eng, 2007. 13(1): p. 111-21.
Noel, D., et al., Cell specific differences between human adipose-derived and mesenchymal-stromal cells despite similar differentiation potentials. Exp Cell Res, 2008. 314(7): p. 1575-84.
Yoo, K. H., et al., Comparison of immunomodulatory properties of mesenchymal stem cells derived from adult human tissues. Cell Immunol, 2009.
Yoshimura, H., et al., Comparison of rat mesenchymal stem cells derived from bone marrow, synovium, periosteum, adipose tissue, and muscle. Cell Tissue Res, 2007. 327(3): p. 449-62.

B. Compositions and Methods

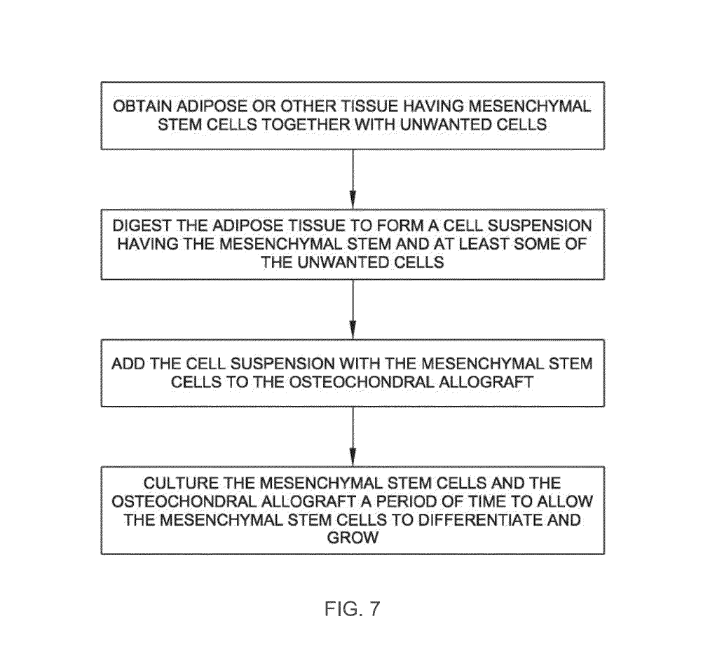

FIG. 7 is a flow chart of a process for combining an osteochondral allograft with stem cells. In an embodiment, a stromal vascular fraction may be used to seed the allograft. It should be apparent from the present disclosure that the term “seed” relates to addition and placement of the stem cells within, or at least in attachment to, the allograft, but is not limited to a specific process.

In an exemplary embodiment, a method of combining mesenchymal stem cells with an osteochondral allograft is provided. The method may include obtaining adipose tissue having the mesenchymal stem cells together with unwanted cells. Unwanted cells may include hematopoietic stem cells and other stromal cells. The method may further include processing (e.g., digesting) the adipose tissue to form a cell suspension having the mesenchymal stem cells and at least some or all of the unwanted cells. In another embodiment, this may be followed by negatively depleting some of the unwanted cells and other constituents to concentrate mesenchymal stem cells.

Next, the method includes adding the cell suspension with the mesenchymal stem cells to seed the osteochondral allograft. This may be followed by allowing the cell suspension to adhere to the osteochondral allograft for a period of time to allow the mesenchymal stem cells to attach. In order to provide a desired product, the method may include rinsing the seeded osteochondral allograft to remove the unwanted cells from the seeded ostechondral allograft.

In one embodiment, an allograft product may include a combination of mesenchymal stem cells with an osteochondral allograft such that the combination is manufactured by the above exemplary embodiment.

In an embodiment, the adipose tissue may be obtained from a cadaveric donor. A typical donor yields 2 liters of adipose containing 18 million MSCs. In one embodiment, an osteochondral allograft may be from the same cadaveric donor as the adipose tissue. In another embodiment, the adipose tissue may be obtained from a patient that will be undergoing the cartilage or osteochondral replacement/regeneration surgery. In addition, both the osteochondral graft (or cartilage or morselized cartilage) and the adipose tissue may be obtained from the same cadaveric donor. Adipose cells may be removed using liposuction. Other sources, and combination of sources, of adipose tissue, other tissues, and osteochondral allografts may be utilized.

Optionally, the adipose tissue may be washed prior to or during processing (e.g., digestion). Washing may include using a thermal shaker at 75 RPM at 37° C. for at least 10 minutes. Washing the adipose tissue may include washing with a volume of PBS substantially equal to the adipose tissue. In an embodiment, washing the adipose tissue includes washing with the PBS with 1% penicillin and streptomycin at about 37° C.

For example, washing the adipose tissue may include agitating the tissue and allowing phase separation for about 3 to 5 minutes. This may be followed by aspirating off a supernatant solution. The washing may include repeating washing the adipose tissue multiple times until a clear infranatant solution is obtained. In one embodiment, washing the adipose tissue may include washing with a volume of growth media substantially equal to the adipose tissue.

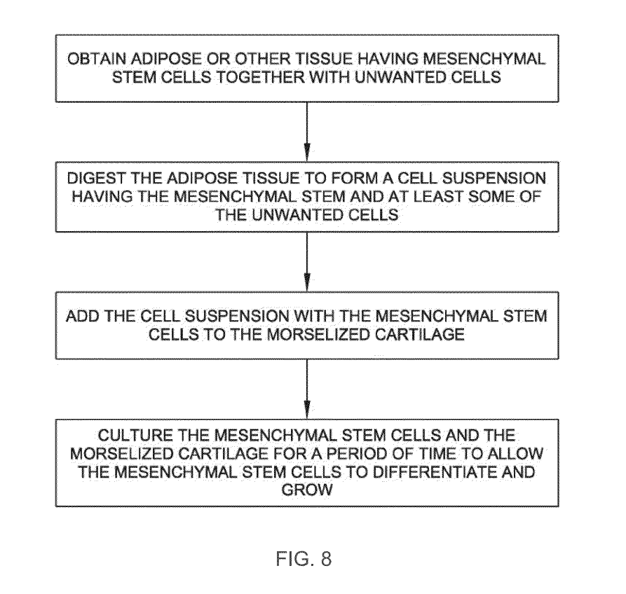

FIG. 8 is a flow chart of a process for combining morselized cartilage with stem cells. In an embodiment, a stromal vascular fraction may be used to seed the allograft.

In another exemplary embodiment, a method of combining mesenchymal stem cells with decellularized, morselized cartilage is provided. The method may include obtaining adipose tissue having the mesenchymal stem cells together with unwanted cells. Unwanted cells may include hematopoietic stem cells and other stromal cells. The method may further include processing (e.g., digesting) the adipose-derived tissue to form a cell suspension having the mesenchymal stem cells and the unwanted cells. In another embodiment, this may be followed by naturally selecting MSCs and depleting some of the unwanted cells and other constituents to concentrate mesenchymal stem cells.

Next, the method includes adding the cell suspension with the mesenchymal stem cells to the morselized cartilage. This may be followed by allowing the cell suspension to adhere to the mesenchymal stem cells and the morselized cartilage for a period of time to allow the mesenchymal stem cells to attach. In order to provide a desired product, the method may include rinsing the seeded morselized cartilage to remove the unwanted cells from the seeded morselized cartilage.

In one embodiment, an allograft product may include a combination of mesenchymal stem cells with decellularized, morselized cartilage such that the combination is manufactured by the above exemplary embodiment.

In an embodiment, the adipose tissue may be obtained from a cadaveric donor. A typical donor yields 2 liters of adipose containing 18 million MSCs. In one embodiment, morselized cartilage may be from the same cadaveric donor as the adipose tissue. In another embodiment, the adipose tissue may be obtained from a patient. In addition, both the osteochondral graft {or cartilage or morselized cartilage) and the adipose tissue may be obtained from the same cadaveric donor. Adipose cells may be removed using liposuction. Other sources, and combination of sources, of adipose tissue, other tissues, and morselized cartilage may be utilized.

Optionally, the adipose tissue may be washed prior to or during processing (e.g., digestion). Washing may include using a thermal shaker at 75 RPM at 37° C. for at least 10 minutes. Washing the adipose tissue may include washing with a volume of PBS substantially equal to the adipose tissue. In an embodiment, washing the adipose tissue includes washing with the PBS with 1% penicillin and streptomycin at about 37° C.

For example, washing the adipose tissue may include agitating the tissue and allowing phase separation for about 3 to 5 minutes. This may be followed by aspirating off a supernatant solution. The washing may include repeating washing the adipose tissue multiple times until a clear infranatant solution is obtained. In one embodiment, washing the adipose tissue may include washing with a volume of growth media substantially equal to the adipose tissue.

Digesting the cell suspension may include making a collagenase I solution, and filtering the solution through a 0.2 μm filter unit, mixing the adipose tissue with the collagenase I solution, and adding the cell suspension mixed with the collagenase 1 solution to a shaker flask. Digesting the cell suspension may further include placing the shaker with continuous agitation at about 75 RPM for about 45 to 60 minutes so as to provide the adipose tissue with a visually smooth appearance.

Digesting the cell suspension may further include aspirating supernatant containing mature adipocytes so as to provide a pellet, which may be referred to as a stromal vascular fraction. (See, for example, FIG. 8.) Prior to seeding, a lab sponge or other mechanism may be used to pat dry cells from the pellet.

In various embodiments, adding the cell suspension with the mesenchymal stem cells to the osteochondral allograft or the morselized cartilage may include using a cell pellet for seeding onto the osteochondral graft (or cartilage or morselized cartilage). In an embodiment, adding the cell suspension with the mesenchymal stem cells to the osteochondral allograft or the morselized cartilage may include using a cell pellet for seeding onto the osteochondral graft (or cartilage or morselized cartilage). In another embodiment, adding the cell suspension with the mesenchymal stem cells to the osteochondral allograft or the morselized cartilage may include using a cell pellet for seeding onto the osteochondral allograft or the morselized cartilage. In another embodiment, adding the cell suspension with the mesenchymal stem cells to seed the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage.

In various embodiments, the method may include placing the osteochondral graft (or cartilage or morselized cartilage) into a cryopreservation media after rinsing the osteochondral allograft or the morselized cartilage. This cryopreservation media may be provided to store the final products. For example, the method may include maintaining the osteochondral allograft or the morselized cartilage into a frozen state after rinsing the osteochondral allograft or the morselized cartilage to store the final products. The frozen state may be at about negative 80° C.

In another embodiment, Ficoll density solution may be utilized. For example, negatively depleting the concentration of the mesenchymal stem cells may include adding a volume of PBS and a volume of Ficoll density solution to the adipose solution. The volume of PBS may be 5 ml and the volume of Ficoll density solution may be 25 ml with a density of 1.073 g/ml. Negatively depleting the concentration of the mesenchymal stem cells may also include centrifuging the adipose solution at about 1160 g for about 30 minutes at about room temperature. In one embodiment, the method may include stopping the centrifuging the adipose solution without using a brake.

Negatively depleting the concentration of the mesenchymal stem cells is optional and may next include collecting an upper layer and an interface containing nucleated cells, and discarding a lower layer of red cells and cell debris. Negatively depleting the concentration of the mesenchymal stem cells may also include adding a volume of D-PBS of about twice an amount of the upper layer of nucleated cells, and inverting a container containing the cells to wash the collected cells. Negatively depleting the concentration of the mesenchymal stem cells may include centrifuging the collected cells to pellet the collected cells using the break during deceleration.

In an embodiment, negatively depleting the concentration of the mesenchymal stem cells may further include centrifuging the collected cells at about 900 g for about 5 minutes at about room temperature. Negatively depleting some of the unwanted cells may include discarding a supernatant after centrifuging the collected cells, and resuspending the collected cells in a growth medium.

In various embodiments, adding the cell suspension with the mesenchymal stem cells to the osteochondral allograft or the morselized cartilage may include adding the cell pellet onto the osteochondral allograft or the morselized cartilage. Adding the solution with the mesenchymal stem cells to the osteochondral allograft or the morselized cartilage may include adding cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In an embodiment, adding the cell suspension with the mesenchymal stem cells to the osteochondral allograft or the morselized cartilage includes adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage. In another embodiment, adding the cell suspension with the mesenchymal stem cells to the osteochondral allograft or the morselized cartilage may include adding the cell pellet onto the osteochondral allograft or the morselized cartilage.

In various embodiments, the method may further include placing the osteochondral allograft or the morselized cartilage into a cryopreservation media after rinsing the osteochondral allograft or the morselized cartilage. This cryopreservation media may be provided to store the final products. The method may include maintaining the osteochondral allograft or the morselized cartilage into a frozen state after rinsing the osteochondral allograft or the morselized cartilage to store the final products. The frozen state may be at about negative 80° C.

The cell suspension is allowed to adhere to seeded allografts for a period of time to allow the mesenchymal stem cells to attach to the osteochondral allograft or the morselized cartilage. The unwanted cells may be rinsed and removed from the osteochondral allograft or the morselized cartilage.

Previous methods used autogenous osteochondral grafts, wherein a graft from one area of a donor knee was transplanted to same donor knee, but to an area that was damaged. However, this method causes trauma to the patient and creates a new area that is damaged. Allografts are currently used that prevent the trauma caused by autografts. Non-processed osteochondral allografts suffer from being immune reactive. Processed osteochondral allografts suffer from either having no viable cells, reduced viability, or fully differentiated cells that are not capable of undergoing regeneration. Thus, there is a need to provide a cartilage graft that contains viable MSCs to recapitulate the regenerative cascade.

The surface of cartilage, by its very nature, is not adherent to cells. The mesenchymal stem cells are anchorage dependent, but this has been defined as being adherent to tissue culture plastic, not a biological tissue like cartilage. Surprisingly, the methods provided herein permit viable MSCs that bind to cartilage.

The methods provided herein describe the allograft processing that allows MSCs to adhere to the scaffold. The method in the example demonstrates a blending and processing method that removes cells from the cartilage graft such that viable MSCs can adhere.

The mesenchymal stem cells are non-immunogenic and regenerate cartilage of the osteochondral allograft or the morselized cartilage. The unwanted cells are generally anchorage independent. This means that the unwanted cells generally do not adhere to the osteochondral allograft or the morselized cartilage. The unwanted cells may be immunogenic. For cell purification during a rinse, mesenchymal stem cells adhere to the osteochondral allograft or the morselized cartilage while unwanted cells, such as hematopoietic stem cells, are rinsed away leaving a substantially uniform population of mesenchymal stem cells on the osteochondral graft (or cartilage or morselized cartilage).

The ability to mineralize the extracellular matrix and to generate cartilage is not unique to MSCs. In fact, ASCs possess a similar ability to differentiate into chondrocytes under similar conditions. Human ASCs offer a unique advantage in contrast to other cell sources. The multipotent characteristics of ASCs, as wells as their abundance in the human body, make these cells a desirable source in tissue engineering applications.

In addition, this method and combination product involve processing that does not alter the relevant biological characteristics of the tissue. Processing of the adipose/stem cells may involve the use of antibiotics, cell media, collagenase. None of these affects the relevant biological characteristics of the stem cells. The relevant biological characteristics of these mesenchymal stem cells are centered on renewal and repair. The processing of the stem cells does not alter the cell's ability to continue to differentiate and repair.

In the absence of stimulation or environmental cues, mesenchymal stem cells (MSCs) remain undifferentiated and maintain their potential to form tissue such as bone, cartilage, fat, and muscle. Upon attachment to an osteoconductive matrix, MSCs have been shown to differentiate along the osteoblastic lineage in vivo. See, for example, the following, which are incorporated by reference:
Arinzeh T. L., Peter S. J., Archambault M. P., van den Bos C., Gordon S., Kraus K., Smith A., Kadiyala S. Allogeneic mesenchymal stem cells regenerate bone in a critical sized canine segmental defect. J Bone Joint Surg Am. 2003; 85-A:1927-35.
Bruder S. P., Kurth A. A., Shea M., Hayes W. C., Jaiswal N., Kadiyala S. Bone regeneration by implantation of purified, culture-expanded human mesenchymal stem cells, J Orthop Res. 1998; 16:155-62.

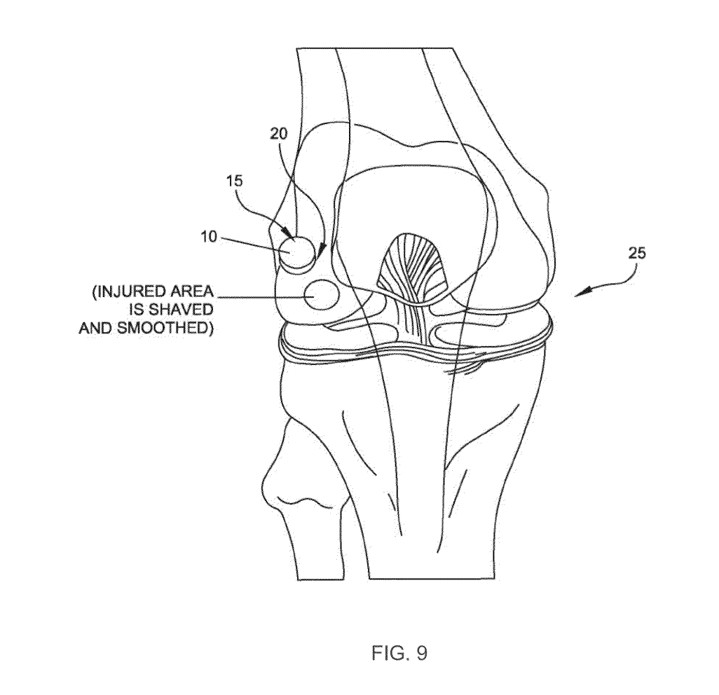

Referring to FIG. 9, and in an embodiment, there is illustrated an osteochondral allograft 10, which may include cartilage 15 and bone 20 from a cadaver. Osteochondral allograft may be placed in the area of a knee 25 or other joint where cartilage is missing. This technique may be used where there is a large area of cartilage that is missing or if there both bone and cartilage are missing. The donor allograft must be tested for contamination, which may include bacteria, hepatitis, and HIV. Having a single donor for both the osteochondral allograft and adipose-derived mesenchymal stem cells may reduce testing burdens and minimize other potential issues.

C. Exemplary Features

In one instance, there is provided a method of combining mesenchymal stem cells with an osteochondral allograft, the method comprising obtaining adipose tissue having the mesenchymal stem cells together with unwanted cells; processing (e.g., digesting) the adipose tissue to form a cell suspension having the mesenchymal stem cells and the unwanted cells; adding the cell suspension with the mesenchymal stem cells to seed the osteochondral allograft so as to form a seeded osteochondral allograft; and allowing the cell suspension to adhere to the osteochondral allograft for a period of time to allow the mesenchymal stem cells to attach.

In some instances, the step of obtaining the adipose tissue includes recovery from a cadaveric donor. In some cases, the osteochondral allograft is from a cadaveric donor, and the step of obtaining the adipose tissue includes recovery from the same cadaveric donor as the osteochondral allograft. In some embodiments, the step of digesting the adipose tissue includes making a collagenase I solution, and filtering the solution through a 0.2 μm filter unit, mixing the adipose with the collagenase I solution, and adding the adipose with the collagenase I solution to a shaker flask. In some instances, the step of digesting the adipose further includes placing the shaker with continuous agitation at about 75 RPM for about 45 to 60 minutes so as to provide the adipose tissue with a visually smooth appearance. In some cases, the step of digesting the adipose further includes aspirating a supernatant containing mature adipocytes so as to provide a pellet. In some instances, the step of adding the suspension with the mesenchymal stem cells to seed the osteochondral allograft includes adding the cell suspension onto the cartilage. In some embodiments, the step of adding the suspension with the mesenchymal stem cells to seed the osteochondral allograft includes adding the cell suspension into decellularized voids in the osteochondral allograft. In some embodiments, the step of adding the suspension with the mesenchymal stem cells to seed the osteochondral allograft includes injecting the suspension into the cartilage. In some instances, the method further comprises removing the unwanted cells from the seeded osteochondral allograft.

In another instance, there is provided an allograft product including a combination of mesenchymal stem cells with an osteochondral allograft, and the combination manufactured by obtaining adipose tissue having the mesenchymal stem cells together with unwanted cells; processing (e.g., digesting) the adipose tissue to form a cell suspension having the mesenchymal stem cells and the unwanted cells; adding the cell suspension with the mesenchymal stem cells to seed the osteochondral allograft so as to form a seeded osteochondral allograft; and allowing the cell suspension to adhere to the seeded osteochondral allograft for a period of time to allow the mesenchymal stem cells to attach.

In another instance, there is provided a method of combining mesenchymal stem cells with an osteochondral allograft, the method comprising obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the osteochondral allograft so as to form a seeded osteochondral allograft; and allowing the cell suspension to adhere to seeded osteochondral allograft for a period of time to allow the mesenchymal stem cells to attach.

In yet another instance, there is provided an allograft product including a combination of mesenchymal stem cells with an osteochondral allograft, and the combination manufactured by obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the osteochondral allograft so as to form a seeded osteochondral allograft; and allowing the cell suspension to adhere to the osteochondral allograft for a period of time to allow the mesenchymal stem cells to attach. In some instances, the adipose tissue is recovered from a cadaveric donor, and the osteochondral allograft is recovered from the same cadaveric donor as the adipose tissue.

In another instance, there is provided a method of combining mesenchymal stem cells with decellularized, morselized cartilage, the method comprising obtaining adipose tissue having the mesenchymal stem cells together with unwanted cells; processing (e.g., digesting) the adipose tissue to form a cell suspension having the mesenchymal stem cells and the unwanted cells; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In some instances, the step of obtaining the adipose tissue includes recovery from a cadaveric donor. In some cases, the morselized cartilage is from a cadaveric donor, and the step of obtaining the adipose tissue includes recovery from the same cadaveric donor as the morselized cartilage. In some instances, the step of digesting the adipose tissue includes making a collagenase I solution, and filtering the solution through a 0.2 μm filter unit, mixing the adipose with the collagenase I solution, and adding the adipose with the collagenase I solution to a shaker flask. In some cases, the step of digesting the adipose further includes placing the shaker with continuous agitation at about 75 RPM for about 45 to 60 minutes so as to provide the adipose tissue with a visually smooth appearance. In some instances, the step of digesting the adipose further includes aspirating a supernatant containing mature adipocytes so as to provide a pellet. In some cases, the step of adding the suspension with the mesenchymal stem cells to seed the morselized cartilage includes adding the cell suspension onto pieces of the morselized cartilage. In some instances, the step of adding the suspension with the mesenchymal stem cells to seed the osteochondral allograft includes adding the cell suspension into voids in the pieces of the morselized cartilage. In some cases, the step of adding the suspension with the mesenchymal stem cells to seed the osteochondral allograft includes injecting the suspension into the pieces of the morselized cartilage. In some instances, the method further comprises removing the unwanted cells from the morselized cartilage.

In another instance, there is provided an allograft product including a combination of mesenchymal stem cells with decellularized, morselized cartilage, and the combination manufactured by obtaining adipose tissue having the mesenchymal stem cells together with unwanted cells; processing (e.g., digesting) the adipose tissue to form a cell suspension having the mesenchymal stem cells and the unwanted cells; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach. In some instances, the adipose tissue is recovered from a cadaveric donor, and the morselized cartilage is recovered from the same cadaveric donor as the adipose tissue

In another instance, there is provided a method of combining mesenchymal stem cells with decellularized, morselized cartilage, the method comprising obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In yet another instance, there is provided an allograft product including a combination of mesenchymal stem cells with decellularized, morselized cartilage, and the combination manufactured by obtaining adipose tissue having the mesenchymal stem cells together with unwanted cells; digesting the adipose tissue to form a cell suspension having the mesenchymal stem cells and the unwanted cells to acquire a stromal vascular fraction, and the digesting includes making a collagenase I solution, and filtering the solution through a 0.2 μm filter unit, mixing the adipose solution with the collagenase I solution, and adding the adipose solution mixed with the collagenase I solution to a shaker flask; placing the shaker with continuous agitation at about 75 RPM for about 45 to 60 minutes so as to provide the adipose tissue with a visually smooth appearance; aspirating a supernatant containing mature adipocytes so as to provide a pellet; adding the cell suspension with the mesenchymal stem cells to seed the morselized cartilage so as to form seeded morselized cartilage; and allowing the cell suspension to adhere to the decellularized, morselized cartilage for a period of time to allow the mesenchymal stem cells to attach. In some instances, the adipose tissue is recovered from a cadaveric donor, and the morselized cartilage is recovered from the same cadaveric donor as the adipose tissue.

In another instance, there is provided a method of combining mesenchymal stem cells with an osteochondral allograft, the method comprising obtaining the mesenchymal stem cells from adipose tissue of a cadaveric donor; obtaining the osteochondral allograft from the same cadaveric donor; adding the mesenchymal stem cells to seed the osteochondral allograft so as to form a seeded osteochondral allograft; and allowing the cell suspension to adhere to the osteochondral allograft for a period of time to allow the mesenchymal stem cells to attach.

In another instance, there is provided an allograft product including a combination of mesenchymal stem cells with an osteochondral allograft, and the combination manufactured by combining mesenchymal stem cells with an osteochondral allograft, the method comprising obtaining the mesenchymal stem cells from adipose tissue of a cadaveric donor; obtaining the osteochondral allograft from the same cadaveric donor; adding the mesenchymal stem cells to seed the osteochondral allograft so as to form a seeded osteochondral allograft; and allowing the cell suspension to adhere to the seeded osteochondral allograft for a period of time to allow the mesenchymal stem cells to attach.

In another instance, there is provided a method of combining mesenchymal stem cells with decellularized, morselized cartilage, the method comprising obtaining the mesenchymal stem cells from adipose tissue of a cadaveric donor; obtaining the morselized cartilage from the same cadaveric donor; adding the mesenchymal stem cells to seed the morselized cartilage so as to form a seeded osteochondral allograft; and allowing the cell suspension to adhere to the decellularized, morselized cartilage for a period of time to allow the mesenchymal stem cells to attach.

In another instance, there is provided an allograft product including a combination of mesenchymal stem cells with decellularized, morselized cartilage, and the combination manufactured by obtaining the mesenchymal stem cells from adipose tissue of a cadaveric donor; obtaining the morselized cartilage from the same cadaveric donor; adding the mesenchymal stem cells to seed the morselized cartilage so as to form seeded morselized cartilage; and allowing the cell suspension to adhere to the decellularized, morselized cartilage for a period of time to allow the mesenchymal stem cells to attach.

In one instance, there is disclosed a method of combining mesenchymal stem cells with cartilage, the method comprising obtaining the mesenchymal stem cells from adipose tissue of a cadaveric donor; obtaining the cartilage from the same cadaveric donor; adding the mesenchymal stem cells to seed the cartilage so as to form a seeded cartilage; and allowing the cell suspension to adhere to the mesenchymal stem cells and the cartilage for a period of time to allow the mesenchymal stem cells to attach.

In another instance, there is disclosed an allograft product including a combination of mesenchymal stem cells with cartilage, and the combination manufactured by obtaining the mesenchymal stem cells from adipose tissue of a cadaveric donor; obtaining the cartilage from the same cadaveric donor; adding the mesenchymal stem cells to seed the cartilage so as to form a seeded cartilage; and allowing the cell suspension to adhere to the mesenchymal stem cells and the cartilage for a period of time to allow the mesenchymal stem cells to attach.

### IV. Collagen Matrix Constructs

A. Introduction

Collagen matrix-containing tissue products, such as small intestinal submucosa, can be applied to a soft tissue injury site to promote repair or reconstruction at the site of injury. It has previously been shown that seeding a collagen matrix-containing tissue product with stem cells promotes more rapid repair or reconstruction than occurs with a non-stem cell seeded collagen matrix tissue product. These results suggest that seeding stem cells on a collagen matrix may promote the rate and/or quality of soft tissue repair or regeneration.

However, previously described stem cell-seeded collagen matrices have utilized stem cells that are grown or proliferated ex vivo (e.g., on a plastic dish) prior to seeding the stem cells on the collagen matrix. Because cell populations change upon attachment to and proliferation on tissue culture plastic, culturing stem cells ex vivo prior to seeding the stem cells on a collagen matrix may result in undesirable phenotypic changes to the seeded stem cells.

Thus, in some embodiments the present invention provides compositions for treating soft tissue injuries comprising a collagen matrix and mesenchymal stem cells adhered to the collagen matrix, wherein the mesenchymal stem cells are derived from a tissue that has been processed (i.e., digested) to form a cell suspension comprising mesenchymal stem cells and non-mesenchymal stem cells that is seeded onto the collagen matrix, and wherein the mesenchymal stem cells are not cultured ex vivo (e.g., on a plastic dish) prior to seeding the cell suspension on the collagen matrix. The present invention also provides for methods of making said compositions comprising a collagen matrix and mesenchymal stem cells adhered to the collagen matrix and methods of treating a subject having a soft tissue injury using said compositions comprising a collagen matrix and mesenchymal stem cells adhered to the collagen matrix.

The present invention also relates to methods of preparing tissues for isolation of cell suspensions comprising mesenchymal stem cells. Cadaveric human tissue is regularly recovered from consented donors to be used in tissue product processing and medical device manufacturing. In some cases, cadaveric tissue may contain certain cell populations, such as progenitor cells or stem cells, which can be incorporated into therapeutic products and methods. Methods for obtaining progenitor cells or stem cells from such tissue are described herein.

In some embodiments, the present invention encompasses systems and methods for the pre-processing of various soft and fibrous tissues, prior to the isolation of progenitor and stem cell populations therefrom. For example, such preparatory techniques can be carried out on the cadaver tissue prior to isolation of the progenitor or stem cells, or prior to isolation of fractions containing such cells. In some cases, preparatory techniques can be performed on adipose tissue, prior to isolation of a stromal vascular fraction (SVF), a progenitor cell population, a stem cell population, or the like. Such isolated cell populations or fractions can be used in therapeutic treatments and products.

B. Compositions for Treating Soft Tissue Injuries

In one aspect, the present invention provides compositions for treating soft tissue injuries, wherein the composition comprises a collagen matrix and mesenchymal stem cells adhered to the collagen matrix. In some embodiments, the mesenchymal stem cells are derived from a tissue that has been processed (i.e., digested) to form a cell suspension comprising mesenchymal stem cells and non-mesenchymal stem cells that is seeded onto the collagen matrix and incubated under conditions suitable for adhering the mesenchymal stem cells to the collagen matrix.

In some embodiments, the mesenchymal stem cells are not cultured ex vivo after formation of the cell suspension and prior to seeding of the cell suspension on the collagen matrix. In some embodiments, the collagen matrix comprises more cells adhered to the outward (epidermal) side or surface of the collagen matrix than to the inward side or surface of the collagen matrix.

1. Collagen Matrix

A collagen matrix for use in the present invention can be from any collagenous tissue. In some embodiments, the collagen matrix is skin, dermis, tendon, ligament, muscle, amnion, meniscus, small intestine submucosa, or bladder. In some embodiments, the collagen matrix is not articular cartilage or bone. In some embodiments, the collagen matrix primarily comprises type I collagen rather than type II collagen.

In some embodiments, the collagen matrix is harvested from a subject, e.g., a human, bovine, ovine, porcine, or equine subject. In some embodiments, the collagen matrix is an engineered collagen matrix, e.g., a matrix that is engineered from one or more purified types of collagen, and optionally further comprising other components commonly found in collagen matrices, e.g., glycosaminoglycans. Engineered collagen matrix is known in the art and is readily commercially available.

In some embodiments, the collagen matrix that is seeded with a cell suspension is a flowable soft tissue matrix. For example, a collagen matrix can be prepared by obtaining a portion of soft tissue material, and cryofracturing the portion of soft tissue material, so as to provide a flowable soft tissue matrix composition upon thawing of the cryofractured tissue. Exemplary compositions and methods involving such flowable matrix materials are described in U.S. patent application Ser. No. 13/712,295, the contents of which are incorporated herein by reference.

In some embodiments, the collagen matrix is allogeneic to the subject in which the collagen matrix is implanted or applied. As non-limiting examples, in some embodiments, the collagen matrix is human and the subject is human, or the collagen matrix is equine and the subject is equine. In some embodiments, the collagen matrix is xenogeneic to the subject in which the collagen matrix is implanted or applied. As a non-limiting example, in some embodiments, the collagen matrix is porcine or bovine and the subject is human. In some embodiments, the collagen matrix is from a cadaveric donor.

In some embodiments, the collagen matrix has low immunogenicity or is non-immunogenic. In some embodiments, the collagen matrix is treated to reduce the immunogenicity of the matrix relative to a corresponding collagen matrix of the same type which has not been treated. Typically, to reduce immunogenicity the collagen matrix is treated to remove cellular membranes, nucleic acids, lipids, and cytoplasmic components, leaving intact a matrix comprising collagen and other components typically associated with the matrix, such as elastins, glycosaminoglycans, and proteoglycans. In some embodiments, immunogenicity of a treated collagen matrix is reduced by at least 50%, at least 60%, at least 70%, at least 80%, at least 90%, or more as compared to an untreated corresponding collagen matrix of the same type (e.g., treated dermis vs. untreated dermis). Any of a number of treatments can be used to reduce the immunogenicity of a collagen matrix, including but not limited to decellularization of the collagen matrix (e.g., by treatment with a surfactant and a protease or nuclease) or cellular disruption of the collagen matrix (e.g., by cryopreservation, freeze/thaw cycling, or radiation treatment). In some embodiments, the collagen matrix is decellularized by treatment with alkaline solution (dilute NaOH) followed by an acid treatment (dilute HCl), resulting in a decellularized neutralized substrate, which can then be submitted to serial washings to remove any remaining water soluble byproducts. Methods of decellularizing or disrupting the cells of a collagen matrix are described, for example, in U.S. Pat. No. 7,914,779; U.S. Pat. No. 7,595,377; U.S. Pat. No. 7,338,757; U.S. Publication No. 2005/0186286; Gilbert et al., J. Surg Res 152:135-139 (2009); and Gilbert et al., Biomaterials 19:3675-83 (2006), the contents of each of which is herein incorporated by reference in its entirety.

The reduction in immunogenicity can be quantified by measuring the reduction in the number of endogenous cells in the treated collagen matrix or by measuring the reduction in DNA content in the treated collagen matrix as compared to a corresponding untreated collagen matrix of the same type, according to methods known in the art. In one non-limiting method, reduction in immunogenicity is quantified by measuring the DNA content of the collagen matrix post-treatment. Briefly, a treated collagen matrix is stained with a fluorescent nucleic acid stain (e.g., PicoGreen® (Invitrogen) or Hoechst 33258 dye), then the amount of fluorescence is measured by fluorometer and compared to the amount of fluorescence observed in a corresponding untreated collagen matrix of the same type which has also been subjected to fluorescent nucleic acid stain. In another non-limiting method, reduction in immunogenicity is quantified by histological staining of the collagen matrix post-treatment using hematoxylin and eosin and optionally DAPI, and comparing the number of cells observed in the treated collagen matrix to the number of cells observed in a corresponding untreated collagen matrix of the same type which has also been subjected to histological staining

In some embodiments, a treated collagen matrix has at least 50%, at least 60%, at least 70%, at least 80%, or at least 90% fewer endogenous cells than a corresponding untreated collagen matrix of the same type. In some embodiments, a treated collagen matrix has a DNA content that is decreased by at least 50%, at least 60%, at least 70%, at least 80%, or at least 90% as compared to a corresponding untreated collagen matrix of the same type.

In some embodiments, the collagen matrix retains bioactive cytokines and/or bioactive growth factors that are endogenous to the collagen matrix. These bioactive cytokines and/or growth factors may enhance or accelerate soft tissue repair or regeneration, for example by recruiting cells to the site of the soft tissue injury, promoting extracellular matrix production, or regulating repair processes. In some embodiments, the collagen matrix retains one or more bioactive cytokines selected from interleukins (e.g., IL-1, IL-4, IL-6, IL-8, IL-15, IL-16, IL-18, and IL-28), tumor necrosis factor alpha (TNFα), and monocyte chemoattractant protein-1 (MCP-1). In some embodiments, the collagen matrix is skin and the one or more bioactive cytokines are selected from IL-4, IL-6, IL-15, IL-16, IL-18, and IL-28. In some embodiments, the collagen matrix is skin and the one or more bioactive cytokines are selected from IL-15 and IL-16. In some embodiments, the collagen matrix retains one or more bioactive growth factors selected from platelet-derived growth factor alpha (PDGFa), matrix metalloproteinase (MMP), transforming growth factor beta (TGFβ), vascular endothelial growth factor (VEGF), and epidermal growth factor (EGF). In some embodiments, the collagen matrix is skin and the one or more bioactive growth factors is PDGFa.

The retention of cytokines and/or growth factors by the collagen matrix, as well as marker profiles of which cytokines and/or growth factors are retained by the collagen matrix, can be determined according to methods known in the art, for example by immunoassay. A variety of immunoassay techniques can be used to detect the presence or level of cytokines and/or growth factors. The term immunoassay encompasses techniques including, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing and Nashabeh, Electrophoresis, 18:2184-2193 (1997); Bao, J. Chromatogr. B. Biomed. Sci., 699:463-480 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, Calif.; Kit #449430) and can be performed using a Behring Nephelometer Analyzer (Fink et al., J. Clin. Chem. Clin. Biol. Chem., 27:261-276 (1989)).

Antigen capture ELISA can be useful for determining the presence or level of cytokines and/or growth factors. For example, in an antigen capture ELISA, an antibody directed to an analyte of interest is bound to a solid phase and sample is added such that the analyte is bound by the antibody. After unbound proteins are removed by washing, the amount of bound analyte can be quantitated using, e.g., a radioimmunoassay (see, e.g., Harlow and Lane, Antibodies: A Laboratory Manual, Cold Spring Harbor Laboratory, New York, 1988)). Sandwich ELISA can also be used. For example, in a two-antibody sandwich assay, a first antibody is bound to a solid support, and the analyte of interest is allowed to bind to the first antibody. The amount of the analyte is quantitated by measuring the amount of a second antibody that binds the analyte. The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

2. Mesenchymal Stem Cells

The mesenchymal stem cells (“MSCs”) which attach to the collagen matrix can be derived from any of a number of different tissues, including but not limited to adipose tissue, muscle tissue, birth tissue (e.g., amnion or amniotic fluid), skin tissue, bone tissue, or bone marrow tissue. The tissue may be harvested from a human subject or a non-human subject (e.g., a bovine, porcine, or equine subject). In some embodiments, the tissue is harvested from a human cadaveric donor. In some embodiments, the tissue is harvested from the subject who is to be treated for a soft tissue injury. In some embodiments, the tissue is allogeneic to the collagen matrix. As non-limiting examples, in some embodiments, the tissue is human and the collagen matrix is human, or the tissue is equine and the collagen maxtrix is equine. In some embodiments, the tissue is xenogeneic to the collagen matrix. As a non-limiting example, in some embodiments, the tissue is human and the collagen matrix is porcine or bovine. In some embodiments, the tissue and the collagen matrix are from the same donor (e.g., the same human donor, e.g., the same cadaveric donor). In some embodiments, the tissue and the collagen matrix are allogeneic but are harvested from different donors (e.g., different human donors, e.g., different cadaveric donors).

In some embodiments, mesenchymal stem cells that are seeded to or that attach to the collagen matrix are identified and characterized based on the presence or absence of one or more markers. In some embodiments, mesenchymal stem cells are identified as having a particular marker profile.

In some embodiments, the mesenchymal stem cells are characterized based on the presence or absence of one, two, three, four, or more markers of cell differentiation (“CD”). In some embodiments, the CD markers are selected from CD34, CD45, CD73, CD90, CD105, CD116, CD144, and CD166. Mesenchymal stem cell markers are described, for example, in Lin et al., Histol. Histopathol. 28:1109-1116 (2013), and in Halfon et al., Stem Cells Dev. 20:53-66 (2011).

As used herein, a “positive” mesenchymal stem cell marker is a marker on the surface of the cell (e.g., a surface antigen, protein, or receptor) that is unique to mesenchymal stem cells. In some embodiments, a positive mesenchymal stem cell marker is CD105, CD144, CD44, CD166, or CD90. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or more of the MSC cells seeded to the collagen matrix are positive for one or more of the CD markers CD105, CD144, CD44, CD166, or CD90.

As used herein, a “negative” mesenchymal stem cell marker is a marker on the surface of the cell (e.g., a surface antigen, protein, or receptor) that is distinctly not expressed by mesenchymal stem cells. In some embodiments, a negative mesenchymal stem cell marker is CD34 or CD116. In some embodiments, at least 25%, at least 30%, at least 35%, at least 40%, at least 45%, at least 50%, at least 55%, at least 60%, at least 65%, at least 70%, at least 75%, or more of the MSC cells seeded to the collagen matrix are negative for one or more of the CD markers CD34 and CD116. In some embodiments, the mesenchymal stem cells are identified as expressing one or more of the positive MSC markers CD105, CD144, CD44, CD166, or CD90 and are further identified as not expressing one or more of the negative MSC markers CD34 and CD116.

The presence and/or amount of a marker of interest on a mesenchymal stem cell can be determined according to any method of nucleic acid or protein expression known in the art. Nucleic acid may be detected using routine techniques such as northern analysis, reverse-transcriptase polymerase chain reaction (RT-PCR), microarrays, sequence analysis, or any other methods based on hybridization to a nucleic acid sequence that is complementary to a portion of the marker coding sequence (e.g., slot blot hybridization). Protein may be detected using routine antibody-based techniques, for example, immunoassays such as ELISA, Western blotting, flow cytometry, immunofluorescence, and immunohistochemistry. In some embodiments, the presence and/or amount of a marker of interest is determined by immunoassay (e.g., ELISA) as described above.

C. Methods of Making Compositions for Treating Soft Tissue Injuries

In another aspect, the present invention provides methods of making a composition for treating a soft tissue injury. In some embodiments, the method comprises: (a) processing (e.g., digesting) a tissue to form a cell suspension comprising mesenchymal stem cells and non-mesenchymal stem cells; (b) seeding the cell suspension onto a collagen matrix; (c) incubating the collagen matrix seeded with the cell suspension under conditions suitable for adhering the mesenchymal stem cells to the collagen matrix; and (d) removing the non-adherent cells from the collagen matrix. In some embodiments, prior to step (b), the method further comprises treating the collagen matrix to reduce the immunogenicity of the collagen matrix.

1. Preparation of a Cell Suspension

A cell suspension comprising mesenchymal stem cells and non-mesenchymal stem cells for seeding onto the collagen matrix can be derived from a variety of types of tissues. In some embodiments, the tissue that is processed (e.g., digested) to form the cell suspension is selected from adipose tissue, muscle tissue, birth tissue (e.g., amnion or amniotic fluid), skin tissue, bone tissue, or bone marrow tissue. In some embodiments, the tissue is harvested from a human subject or a non-human subject (e.g., a bovine, porcine, or equine subject). In some embodiments, the tissue is harvested from a human cadaveric donor. In some embodiments, the tissue is harvested from the subject who is to be treated for a soft tissue injury.

Exemplary methods of forming a cell suspension from tissue by enzymatic digestion and seeding the cell suspension onto a scaffold are described herein for adipose tissue. A tissue may be enzymatically digested to form a cell suspension comprising mesenchymal stem cells and unwanted cells. In some embodiments, the tissue is digested with a collagenase solution (e.g., collagenase I). Optionally, the tissue is digested with the collagenase solution under continuous agitation (e.g., at about 75 rpm) for a suitable period of time (e.g., 30 minutes, 45 minutes, 60 minutes, or longer) until the tissue appears smooth by visual inspection.

Optionally, the tissue may be washed prior to or during digestion (processing). In some embodiments, the tissue is washed with a volume of a solution (e.g., phosphate-buffered saline (PBS) or growth media) that is at least substantially equal to the tissue. In some embodiments, the tissue is washed with a solution comprising antibiotics (e.g., 1% penicillin and streptomycin) and/or antimycotics. In some embodiments, the tissue is washed at about 37° C., optionally with shaking to agitate the tissue. Washing may include repeated steps of washing the tissue, then aspirating off a supernatant tissue, then washing with fresh solution, until a clear infranatant solution is obtained.

Digestion of the tissue followed by centrifugation of the digested tissue results in the formation of a cell suspension, which can be aspirated to remove the supernatant and leave a cell pellet comprising mesenchymal stem cells and unwanted cells. The cell pellet is resuspended in a solution (e.g., growth media with antibiotics) and the resulting cell suspension is then seeded on a collagen matrix without any intervening steps of further culturing or proliferating the mesenchymal stem cell-containing cell suspension prior to the seeding step.

In some embodiments, the cell suspension can be enriched for stem cells by serial plating on a collagen-coated substrate prior to seeding the cell suspension on the collagen matrix. As one non-limiting example, muscle tissue can be prepared according to the following method to form an enriched cell suspension for seeding on a collagen matrix. The harvested muscle sample is minced, digested at 37° C. with 0.2% collagenase, trypsinized, filtered through 70 μm filters, and cultured in collagen-coated cell culture dishes (35-mm diameter, Corning, Corning, N.Y.) at 37° C. in F12 medium (Gibco, Paisley, UK), with 15% fetal bovine serum. After a suitable period of time (e.g., one hour), the supernatant is withdrawn from the cell culture dishes and re-plated in fresh collagen-coated cell culture dishes. The cells that adhere rapidly within this time period will be mostly unwanted cells (e.g., fibroblasts). When 30%-40% of the cells have adhered to each collagen-coated cell culture dish, serial re-plating of the supernatant is repeated. After 3-4 serial re-platings, the culture medium is enriched with small, round cells, thus forming a stem cell-enriched cell suspension.

2. Seeding the Collagen Matrix

For seeding the cell suspension onto the collagen matrix, the collagen matrix may be placed in a culture dish, e.g., a 24-well culture plate and then the cell suspension added onto the collagen matrix. The collagen matrix onto which the cell suspension is seeded can be any collagen matrix as described herein. In some embodiments, the collagen matrix is skin, dermis, tendon, ligament, muscle, amnion, meniscus, small intestine submucosa, or bladder. In some embodiments, the collagen matrix is not articular cartilage. In some embodiments, wherein the collagen matrix comprises multiple layers, one or more of the matrix layers can be seeded with the cell suspension. As a non-limiting example, in some embodiments a dermal matrix comprises two layers, an epidermal facing basement membrane and a deeper hypodermal surface. The cell suspension can be seeded on the epidermal facing basement membrane, the deeper hypodermal surface, or both the epidermal facing basement membrane and the deeper hypodermal surface.

In some embodiments, the collagen matrix is treated to reduce immunogenicity prior to seeding the cell suspension on the collagen matrix. In some embodiments, the immunogenicity of the collagen matrix after treatment is reduced by at least 50%, at least 60%, at least 70%, at least 80%, at least 90%, or more as compared to an untreated corresponding collagen matrix of the same type. In some embodiments, the treated collagen matrix is non-immunogenic. As described above, any of a number of treatments can be used to reduce the immunogenicity of a collagen matrix, including but not limited to decellularization of the collagen matrix (e.g., by treatment with a surfactant and a protease or nuclease) or cellular disruption of the collagen matrix (e.g., by cryopreservation, freeze/thaw cycling, or radiation treatment). In some embodiments, the collagen matrix is treated with a decellularizing agent (e.g., a solution comprising a surfactant and a protease or a surfactant and a nuclease). Other suitable methods of decellularization are described in Crapo et al., Biomaterials 32:3233-43 (2011), the contents of which are incorporated by reference herein.

Following seeding of the cell suspension onto the collagen matrix, the cell suspension-seeded collagen matrix is incubated under conditions suitable for adhering mesenchymal stem cells to the matrix. In some embodiments, the cell suspension-seeded collagen matrix is incubated for several days (e.g., up to about 24 hours, about 36 hours, about 48 hours, about 60 hours, or about 72 hours) to allow adherence. In some embodiments, the cell suspension-seeded collagen matrix is incubated in a CO₂ incubator at about 37° C. The cell suspension-seeded collagen matrix may be incubated with culture medium (e.g., DMEM/F12), optionally with supplements and/or antibiotics and/or antimycotics (e.g., DMEM/F12 with 10% fetal bovine serum (FBS) and 1% penicillin, streptomycin, and amphotericin B (PSA)). In some embodiments, a greater number of mesenchymal stem cells adhere to the outward (epidermal) side or surface of the collagen matrix than to the inward (hypodermal) side or surface of the collagen matrix.

After the incubation step, the cell suspension-seeded collagen matrix is washed (e.g., with PBS or culture medium) to remove non-adherent cells from the collagen matrix. In some embodiments, the collagen matrix with adherent mesenchymal stem cells is placed in cryopreservation media (e.g., 10% DMSO, 90% serum) and kept frozen at −80° C.

3. Preparation of Tissues for Isolation of Cell Suspension

In some embodiments, the present invention provides techniques for manipulating large quantities or volumes of adipose, muscle, and other soft and fibrous tissues containing progenitor and stem cell populations, in a repeatable and consistent manner, by mechanical grinding to a defined particle size, in order to effectively prepare the tissues for isolation of a cell suspension (e.g., the stromal vascular fraction (SVF) of adipose tissue), prior to processing (including enzymatic or other digestion techniques).

Exemplary methods may include preparing large pieces and large quantities of adipose, muscle, or other tissues containing progenitor or stem cell populations, or both, for isolation of a cell suspension using a repeatable and consistent method of grinding, which can be applied to large-scale use. In this way, large pieces and large amounts of tissue can be efficiently broken down into a form suitable for subsequent isolation of the cell suspension using enzymatic or other digestion techniques or other methods. The use of mechanical grinding can enhance consistency and reproducibility through engineering controls.

In some instances, embodiments are directed toward the preparation of cadaveric tissues for optimal isolation of the cell suspension, in terms of large scale efficiency. Adipose or other tissue types are recovered from donor cadavers and transported to a processing facility. The tissue is repeatedly washed in Dulbecco's Phosphate-Buffered Saline (DPBS) or another isotonic reagent, optionally with antibiotic and/or antimycotic solution, to remove blood and other debris. The tissue is then ground from its original large size into small, consistent particles. The reduced particle size and increased surface area allow for more efficient processing (including digestion, by enzymes or other techniques), and improved yield of the progenitor and stem cell-containing cell suspension. The small particles can then be washed again in isotonic solution, such as DPBS.

In some embodiments, it may be useful to rinse the tissue, either before grinding, after grinding, or both. Specific rinsing protocols can be selected to achieve a desired result, and may be performed in any combination. For example, a final cell population may be affected by the number of rinses and the sequence in which the tissue is ground and rinsed. Therefore, embodiments of the present invention encompass techniques which involve rinsing before grinding, rinsing after grinding, and rinsing before and after grinding, and the selected technique may depend on the desired cell population.

The grinding protocols disclosed herein may provide enhanced results when compared to certain currently known techniques. For example, some known techniques involve enzymatically digesting large pieces of tissue, such as adipose tissue, in their originally harvested form. Relatedly, some known techniques are limited to the isolation of a cell suspension in only very small amounts (e.g., ˜50 cc), for example using recovered lipoaspirate, whole pieces, or hand-minced particles.

In contrast, embodiments of the present invention facilitate large-scale manufacturing techniques using large amounts of tissue which can be processed in a timely and consistent manner. Toward this end, a mechanical grinder can be used to reduce harvested tissue into smaller particles to promote efficiency of isolation of a cell suspension for large scale manufacturing. In some aspects, such reduction of the particle size provides an increased surface area and allows quicker, more efficient processing (e.g., digestion) and isolation of the cell suspension. According to some embodiments, a mechanical grinder can be used to process the harvested tissue into particles having uniform sizes and shapes. In some embodiments, the process is automated so that tissue pieces having uniform size or shape properties can be obtained regardless of any subjectivity on the part of the operator.

In some embodiments, a standard grinder is used to reduce particle size consistently for large scale, regulated operations. Components of an exemplary grinding apparatus can be made of durable, autoclavable, and inert materials, such as stainless steel, which may facilitate ease of use and withstand large scale manufacturing workloads. In some cases, a grinding system can be manually operated. In some case, a grinding system can be electrically operated. The tissue types processed by the grinding system may include any soft tissues containing progenitor and stem cell populations such as adipose, muscle, skin, birth tissues, and the like. Various grinder speeds and attachments can be used to break down the tissue to a preferred particle size for each specific tissue type or application.

The tissue pre-processing systems and methods disclosed herein are well suited for use with the large scale production of tissue and medical devices involving large amounts of stem and progenitor cells. In accordance with these techniques, the donor cell yield can be maximized. In some cases, the grinding approaches can be utilized on the front end of the process, whereby soft/fibrous tissues are recovered from donor cadavers in bulk and ground at a processing facility to yield large amounts of cell suspensions comprising stem or progenitor cell populations. In some cases, tissue harvesting techniques may provide recovered tissue in large pieces and in large amounts. Relatedly, adipose processing techniques disclosed herein may be used as a primary method of large scale adipose recovery, which may optionally be supplanted by liposuction.

D. Methods of Treatment

In yet another aspect, the present invention provides methods of treating a soft tissue injury in a subject using a composition as described herein (e.g., a composition comprising a collagen matrix and mesenchymal stem cells adhered to the collagen matrix). In some embodiments, the method comprises contacting a soft tissue injury site with a composition as described herein.

The compositions of the present invention can be used to treat subjects having any soft tissue injury that requires repair or regeneration. Such soft tissue injuries may result, for example, from disease, trauma, or failure of the tissue to develop normally. Examples of soft tissue injuries that can be treated according to the methods of the present invention include, but are not limited to, tears or ruptures of a soft tissue (e.g., tendon, ligament, meniscus, muscle, bladder or skin); hernias; skin wounds; burns; skin ulcers; surgical wounds; vascular disease (e.g., peripheral arterial disease, abdominal aortic aneurysm, carotid disease, and venous disease; vascular injury; improper vascular development); and muscle diseases (e.g., congenital myopathies; myasthenia gravis; inflammatory, neurogenic, and myogenic muscle diseases; and muscular dystrophies such as Duchenne muscular dystrophy, Becker muscular dystrophy, myotonic dystrophy, limb-girdle-muscular dystrophy, facioscapulohumeral muscular dystrophy, congenital muscular dystrophies, oculopharyngeal muscular dystrophy, distal muscular dystrophy, and Emery-Dreifuss muscular dystrophy). In some embodiments, the soft tissue injury is an injury to a tendon tissue, a ligament tissue, a meniscus tissue, a muscle tissue, a skin tissue, a bladder tissue, or a dermal tissue. In some embodiments, the soft tissue injury is a surgical wound, a trauma wound, a chronic wound, an acute wound, a deep channel wound, an exsanguinating site, or a burn.

In some embodiments, the composition is allogeneic to the subject that is being treated. As non-limiting examples, in some embodiments, the collagen matrix is human, the mesenchymal stem cells adhered to the matrix are human, and the subject is human; or the collagen matrix is equine, the mesenchymal stem cells adhered to the matrix are equine, and the subject is equine. In some embodiments, the composition is xenogeneic to the subject that is being treated. As a non-limiting example, in some embodiments, the collagen matrix is porcine or bovine, the mesenchymal stem cells adhered to the matrix are human, and the subject is human.

In some embodiments, the compositions described herein are used to treat humans having a soft tissue injury as described above. In some embodiments, the compositions described herein are used for veterinary applications. For example, in some embodiments, a composition of the present invention is used a non-human animal such as a non-human primate, mouse, rat, dog, cat, pig, sheep, cow, or horse having a soft tissue injury as described above. In some embodiments, a composition as described herein is used to treat a horse having a ruptured or torn soft tissue (e.g., ligament).

A mesenchymal stem cell-seeded collagen matrix of the present invention can be applied or introduced into a subject's body according to any method known in the art, including but not limited to implantation, injection, topical application, surgical attachment, or transplantation with other tissue. In some embodiments, the composition is administered topically. In some embodiments, the composition is administered by surgical implantation. The matrix may be configured to the shape and/or size of a tissue or organ or can be resized prior to administration (e.g., by a surgeon) to the size of the soft tissue injury being repaired. In some embodiments, a mesenchymal stem cell-seeded collagen matrix of the present invention is multilayered.

E. Exemplary Features

In one instance, this disclosure provides compositions for treating a soft tissue injury in a subject. In some embodiments, the composition comprises a collagen matrix and mesenchymal stem cells adhered to the collagen matrix, wherein the mesenchymal stem cells are derived from a tissue processed to form a cell suspension comprising mesenchymal stem cells and non-mesenchymal stem cells that is seeded onto the collagen matrix, and wherein the mesenchymal stem cells are not cultured ex vivo after formation of the cell suspension and prior to seeding of the cell suspension on the collagen matrix.

In some instances, the collagen matrix is skin, dermis, tendon, ligament, muscle, amnion, meniscus, small intestine submucosa, or bladder. In some instances, the collagen matrix is decellularized dermis. In some instances, the collagen matrix is dermis from which the epidermis layer has been removed.

In some instances, the collagen matrix is treated to reduce immunogenicity. In some embodiments, the treated collagen matrix has at least 50% fewer endogenous cells than a corresponding untreated collaged matrix of the same type. In some cases, the treated collagen matrix has a DNA content that is decreased by at least 50% as compared to a corresponding untreated collaged matrix of the same type. In some cases, the treated collagen matrix is non-immunogenic.

In some instances, the treated collagen matrix retains bioactive cytokines. In some embodiments, the bioactive cytokines are selected from the group consisting of IL-4, IL-6, IL-15, IL-16, IL-18, and IL-28. In some embodiments, the treated collagen matrix retains bioactive growth factors. In some embodiments, the bioactive growth factor is platelet-derived growth factor alpha (PDGFa).

In some cases, the collagen matrix is human, porcine, bovine, or equine.

In some cases, the tissue that is processed to form the cell suspension is selected from adipose tissue, muscle tissue, birth tissue, skin tissue, bone tissue, or bone marrow tissue. In some embodiments, the tissue that is processed to form the cell suspension is human tissue.

In some instances, the collagen matrix and the tissue that is processed to form the cell suspension are from the same species. In some embodiments, the collagen matrix and the tissue that is processed to form the cell suspension are from different species. In some embodiments, the collagen matrix and the tissue that is processed to form the cell suspension are from the same donor. In some embodiments, the collagen matrix and the tissue that is processed to form the cell suspension are from different cadaveric donors. In some embodiments, the donor is human.

In some instances, mesenchymal stem cells seeded on the collagen matrix express one or more of the positive MSC markers CD105, CD144, CD44, CD166, or CD90. In some embodiments, mesenchymal stem cells seeded on the collagen matrix do not express one or more of the negative MSC markers CD34 and CD116.

In another instance, this disclosure provides methods of treating a soft tissue injury in a subject. In some embodiments, the method comprises contacting a composition as described herein (e.g., a composition comprising a collagen matrix and mesenchymal stem cells adhered to the collagen matrix, wherein the mesenchymal stem cells are derived from a tissue processed to form a cell suspension comprising mesenchymal stem cells and non-mesenchymal stem cells that is seeded onto the collagen matrix, and wherein the mesenchymal stem cells are not cultured ex vivo after formation of the cell suspension and prior to seeding of the cell suspension on the collagen matrix) to the site of the soft tissue injury.

In some cases, the soft tissue injury is an injury to a tendon tissue, a ligament tissue, a meniscus tissue, a muscle tissue, a skin tissue, a bladder tissue, or a dermal tissue. In some embodiments, the soft tissue injury is a surgical wound, a trauma wound, a chronic wound, an acute wound, a deep channel wound, an exsanguinating site, or a burn.

In some cases, the composition is administered topically. In some embodiments, the composition is administered by surgical implantation.

In some instances, the subject is a human subject. In some embodiments, the subject is a veterinary subject. In some embodiments, the veterinary subject is a horse.

In another instance, this disclosure provides methods of making a composition for treating a soft tissue injury. In some embodiments, the method comprises: (a) processing (e.g., digesting) a tissue to form a cell suspension comprising mesenchymal stem cells and non-mesenchymal stem cells; (b) seeding the cell suspension onto a collagen matrix; (c) incubating the collagen matrix seeded with the cell suspension under conditions suitable for adhering the mesenchymal stem cells to the collagen matrix; and (d) removing the non-adherent cells from the collagen matrix.

In some cases, prior to step (b), the method further comprises treating the collagen matrix to reduce immunogenicity. In some instances, treating the collagen matrix to reduce immunogenicity comprises contacting the collagen matrix with a decellularizing agent. In some instances, treating the collagen matrix to reduce immunogenicity comprises removing an epidermis layer without decellularizing the collagen matrix. In some cases, the treated collagen matrix has at least 50% fewer endogenous cells than a corresponding untreated collaged matrix of the same type. In some cases, the treated collagen matrix has a DNA content that is decreased by at least 50% as compared to a corresponding untreated collaged matrix of the same type. In some instances, the treated collagen matrix is non-immunogenic.

In some instances, the method further comprises a washing step to remove the decellularizing agent. In some cases, the washing step is performed after decellularization and before the cell suspension is seeded on the collagen matrix.

In some instances, the collagen matrix is skin, dermis, tendon, ligament, muscle, amnion, meniscus, small intestine submucosa, or bladder.

In some cases, the treated collagen matrix retains bioactive cytokines. In some embodiments, the bioactive cytokines are selected from the group consisting of IL-4, IL-6, IL-15, IL-16, IL-18, and IL-28. In some instances, the treated collagen matrix retains bioactive growth factors. In some embodiments, the bioactive growth factor is platelet-derived growth factor alpha (PDGFa).

In some instances, the collagen matrix is human, porcine, bovine, or equine.

In some cases, the tissue that is processed (e.g., digested) to form the cell suspension is selected from adipose tissue, muscle tissue, birth tissue, skin tissue, bone tissue, or bone marrow tissue. In some embodiments, the tissue that is processed (e.g., digested) to form the cell suspension is human tissue.

In some cases, the collagen matrix and the tissue that is processed (e.g., digested) to form the cell suspension are from the same species. In some embodiments, the collagen matrix and the tissue that is processed (e.g., digested) to form the cell suspension are from different species. In some embodiments, the collagen matrix and the tissue that is processed (e.g., digested) to form the cell suspension are from the same donor. In some embodiments, the collagen matrix and the tissue that is processed (e.g., digested) to form the cell suspension are from different cadaveric donors. In some embodiments, the donor is human.

### Examples

The following examples are offered to illustrate, but not to limit the claimed invention.

### A. Bone Construct Examples

### Example A1

### a. Adipose Recovery

Adipose was recovered from cadaveric donors. Adipose aspirate may be collected using liposuction machine and shipped on wet ice.

### b. Washing

Adipose tissue was warmed up in a thermal shaker at RPM=75, 37° C. for 10 min. Adipose was washed with equal volume of pre-warmed phosphate buffered saline (PBS) at 37° C., 1% penicillin/streptomycin. Next, the adipose was agitated to wash the tissue. Phase separation was allowed for about 3 to 5 minutes. The infranatant solution was aspirated. The wash was repeated 3 to 4 times until a clear infranatant solution was obtained.

The solution was suspended in an equal volume of growth media (DMEM/F12, 10% FBS, 1% penicillin/streptomycin) and stored in a refrigerator at about 4° C.

### c. Digestion and Combining of Cell Suspension with Allografts

Digestion of the adipose was undertaken to acquire a stromal vascular fraction (SVF) followed by combining the solution onto an allograft.

Digestion involved making collagenase I solution, including 1% fetal bovine serum (FBS) and 0.1% collagenase I. The solution was filtered through a 0.2 urn filter unit. This solution should be used within 1 hour of preparation.

Next, take out the washed adipose and mix with collagenase I solution at 1:1 ratio. Mixture was added to a shaker flask.

The flask was placed in an incubating shaker at 37° C. with continuous agitation (at about RPM=75) for about 45 to 60 minutes until the tissue appeared smooth on visual inspection.

The digestate was transferred to centrifuge tubes and centrifuged for 5 minutes at about 300-500 g at room temperature. The supernatant, containing mature adipocytes, was then aspirated. The pellet was identified as the stromal vascular fraction (SVF).

Growth media was added into every tube (i.e., 40 ml total was added into the 4 tubes) followed by gentle shaking.

All of the cell mixtures were transferred into a 50 ml centrifuge tube. A 200 μl sample was taken, 50 μl is for initial cell count, and the remainder of the 150 μl was used for flow cytometry.

Aliquot cell mixtures were measured into 2 centrifuge tubes (of 10 ml each) and centrifuged at about 300 g for 5 minutes. The supernatant was aspirated.

A cell pellet obtained from one tube was used for seeding onto allografts. The allografts may include cortical/cancellous or both which was subjected to a demineralization process.

Certain volume of growth medium was added into the cell pellets and shaken to break the pellets. A very small volume of cell suspension was added onto allografts. After culturing in CO₂ incubator at 37° C. for a few hours, more growth medium (DMEM/F12, 10% FBS with antibiotics) was added. This was astatic “seeding” process. A dynamic “seeding” process can be used for particular bone substrate. 10 ml of a cell suspension and bone substrate were placed in a 50 ml centrifuge tube on an orbital shaker and agitated at 100 to 300 rpm for 6 hours.

After a few days (about 1 to 3 days), the allograft was taken out and rinsed thoroughly in PBS and sonicated to remove unwanted cells. The allograft was put into cryopreservation media (10% DMSO, 90% serum) and kept frozen at −80° C. The frozen allograft combined with the mesenchymal stem cells is a final product.

### Example A2

### a. Adipose Recovery

Adipose was recovered from cadaveric donors. Adipose aspirate may be collected using liposuction machine and shipped on wet ice.

### b. Washing

Adipose tissue was processed in a thermal shaker at RPM=75, 37° C. for 10 min. Adipose was washed with equal volume of pre-warmed phosphate buffered saline (PBS) at 37° C., 1% penicillin/streptomycin. Next, the adipose was agitated to wash the tissue. Phase separation was allowed for about 3 to 5 minutes. The supernatant solution was sucked off. The wash was repeated 3 to 4 times until a clear infranatant solution was obtained.

### c. Acquire Ficoll Concentrated Stem Cells and Combine onto Allograft

Ficoll concentrated stem cells were acquired and seeded onto an allograft. 5 ml PBS was placed into the 50 ml tube with cells and 25 ml of 1.073 g/ml Ficoll density solution was added to the bottom of the tube with a pipet.

The tubes were subjected to centrifugation at 1160 g for 30 min at room temperature and stopped with the brake off. The upper layer and interface, approximately 15 to 17 ml containing the nucleated cells were collected with a pipet and transferred to a new 50 ml disposable centrifuge tube. The lower layer contained red cells and cell debris and was discarded.

Next, 2 volumes of 0-PBS were added. The tubes were capped and mix gently by inversion to wash the cells.

The tubes with the diluted cells were then subjected to centrifugation at 900 g for 5 minutes at room temperature to pellet the cells with the brake on during deceleration.

The supernatant was discarded and the washed cells were resuspended in 10 ml of growth medium. 10 ml of growth media was added into the tube and it was shaken gently. A 1 ml sample was taken with 100 pl is for cell count, and the remainder of 900 μl was used for flow cytometry.

The remainder of the cell mixtures were centrifuged at about 300 g for about 5 minutes. The supernatant was aspirated.

A cell pellet was used for “seeding” onto allografts. Allografts may include demineralized bone, cortical/cancellous bone, or both. A very small volume of medium was added into the cell pellet and shaken. 100 μl of cell mixtures were added onto a 15 mm disc within a 24-well culture plate.

After culturing the allograft in a CO₂ incubator at about 37° C., 1 ml growth medium (DMEM/F12, 10% FBS with antibiotics) was added. This was a static “seeding” process. A dynamic “seeding” process can be used for a particular bone substrate.

After a few days (about 1 to 3 days), the allograft was taken out and rinsed thoroughly in PBS to remove unwanted cells. The allograft was put into cryopreservation media (10% DMSO, 90% serum) and kept frozen at −80° C. The frozen allograft combined with the stem cells is a final product.

### Example A3

### a. Bone Marrow Recovery

Bone marrow was recovered from cadaveric donors and shipped on wet ice.

### b. Washing

The bone marrow sample is washed by adding 6 to 8 volumes of Dulbecco's phosphate buffered saline (D-PBS) in a 50 ml disposable centrifuge, inverting gently and subjecting to centrifugation (800 g for 10 min) to pellet cells to the bottom of the tube.

### c. Acquire Stem Cells and Combine onto Allograft

The supernatant is discarded and the cell pellets from all tubes are resuspended in 1-2 ml of growth medium (DMEM, low glucose, with 10% FBS and 1% pen/strap). The cell mixtures are seeded onto allografts. With a few hours of culture in CO₂ incubator at 37° C., more growth medium is added. A few days later, the allograft is taken out and rinsed thoroughly in PBS and put into cryopreservation media (10% DMSO, 90% serum) and kept frozen.

### Example A4

### a. Skeletal Muscle Recovery

Skeletal muscle may be recovered from cadaveric donors.

### b. Washing

Minced skeletal muscle (1-3 mm cube) is digested in a 3 mg/ml collagenase D solution in α-MEM at 37° C. for 3 hours. The solution is filtered with 100 um nylon mesh. The solution is centrifuged at 500 g for 5 min.

### c. Acquire Stem Cells and Combine onto Allograft

The supernatant is discarded and the cell pellets from all tubes are resuspended in 1-2 ml of growth medium (DMEM, low glucose, with 10% FBS and 1% pen/strap). The cell mixtures are seeded onto allografts. With a few hours of culture in CO₂ incubator at 37° C., more growth medium will be added. A few days later, the allograft is taken out and rinsed thoroughly in PBS and put into cryopreservation media (10% DMSO, 90% serum) and kept frozen.

### Example A5

### a. Adipose Recovery

Adipose was recovered from a cadaveric donor within 24 hours of death and shipped in equal volume of DMEM in wet ice.

### b. Washing

Adipose were washed 3 times with PBS and suspended in an equal volume of PBS supplemented with Collagenase Type I pre-warmed to 37° C. The tissue was placed in a shaking water bath at 37° C. with continuous agitation for 45 to 60 minutes and centrifuged for 5 minutes at room temperature. The supernatant, containing mature adipocytes, was aspirated. The pellet was identified as the SVF (stromal vascular fraction).

### c. Cortical Cancellous Bone Recovery

Human cortical cancellous bone was recovered from ilium crest from the same donor. The samples were sectioned into strips (20×50×5 mm), and then they were subjected to a demineralization process with HCl for 3 hours, rinsed with PBS until the pH is neutral.

### d. Digestion and Combining of Cell Suspension with Allograft

The adipose-derived stem cells (ASCs) were added onto the grafts and cultured in CO₂ incubator at 37° C. Then the allografts were rinsed thoroughly in PBS to remove antibiotics and other debris. At the end, the allografts were put into cryopreservation media and kept frozen at −80° C.

### Example A6

### a. Adipose-Derived Stem Cell Characterization

i. Flow Cytometry Analysis

The following antibodies were used for flow cytometry. PE anti-CD73 (clone AD2) Becton Dickinson, PE anti-CD90 (clone F15-42-1) AbD SeroTec, PE anti-CD105 (clone SN6) AbD SeroTec, PE anti-Fibroblasts/Epithelial Cells (clone 07-FIB) AbD SeroTec, FITC anti-CD34 (clone 8G12) Becton Dickinson, FITC Anti-CD45 (clone 2D1) Becton Dickinson, and PE anti-CD271 (clone ME20.4-1.H4) Miltenyi BioTec. The Isotype controls were FITC Mouse IgG1 Kappa (clone MOPC-21) Becton Dickinson, PE Mouse IgG1 Kappa (clone MOPC-21) Becton Dickinson, and PE Mouse IgG2a Kappa (clone G155-178) Becton Dickinson.

A small aliquot of the cells were stained with a propidium iodide/detergent solution and fluorescent nuclei were counted using a hemocytometer on a fluorescent microscope. This total cell count was used to adjust the number of cells per staining tube to no more than 5.0×105 cells. The cells were washed with flow cytometric wash buffer (PBS supplemented with 2% FBS and 0.1% NaN3), stained with the indicated antibodies and washed again before acquisition. Staining was for 15 minutes at room temperature (15-30DC).

At least 20,000 cells were acquired for each sample on a FACScan flow cytometer equipped with a 15-mW, 488-nm, argon-ion laser (BD Immunocytometry Systems, San Jose, Calif.). The cytometer QC and setup included running SpheroTech rainbow (3 μm, 6 peaks) calibration beads (SpheroTech Inc.) to confirm instrument functionality and linearity. Flow cytometric data were collected and analyzed using Cell Quest software (BD Immunocytometry Systems). The small and large cells were identified by forward (FSC) and side-angle light scatter (SSC) characteristics. Autofluorescence was assessed by acquiring cells on the flow cytometer without incubating with fluorochrome labeled antibodies. Surface antigen expression was determined with a variety of directly labeled antibodies according to the supplier's recommendations. Antibodies staining fewer than 20% of the cells relative to the Isotype-matched negative control were considered negative (this is standard-of-practice for immunophenotyping leukocytes for leukemia lymphoma testing). The viability of the small and large cells was determined using the Becton Dickinson Via-Probe (7-AAD).

ii. In Vitro Tri-Lineage Differentiation

Osteogenesis—Confluent cultures of primary ASCs were induced to undergo osteogenesis by replacing the stromal medium with osteogenic induction medium (Stempro® osteogenesis differentiation kit, Invitrogen). Cultures were fed with fresh osteogenic induction medium every 3 to 4 days for a period of up to 3 weeks. Cells were then fixed in 10% neutral buffered formalin and rinsed with Dl water. Osteogenic differentiation was determined by staining for calcium phosphate with Alizarin red (Sigma).

Adipogenesis—Confluent cultures of primary ASCs were induced to undergo adipogenesis by replacing the stromal medium with adipogenic induction medium (Stempro® adipogenesis differentiation kit, Invitrogen). Cultures were fed with fresh adipogenic induction medium every 3 to 4 days for a period of up to 2 weeks. Cells were then fixed in 10% neutral buffered formalin and rinsed with PBS. Adipogenic differentiation was determined by staining for fat globules with oil red 0 (Sigma).

Chondrogenesis—Confluent cultures of primary ASCs were induced to undergo chondrogenesis by replacing the stromal medium with chondrogenic induction medium (Stempro® chondrogenesis differentiation kit, Invitrogen). Cultures were fed with fresh chondrogenic induction medium every 3 to 4 days for a period of up to 3 weeks. Cells were then fixed in 10% neutral buffered formalin and rinsed with PBS. Chondrogenic differentiation was determined by staining for proteoglycans with Alcian blue (Sigma).

iii. Final Product Characterization

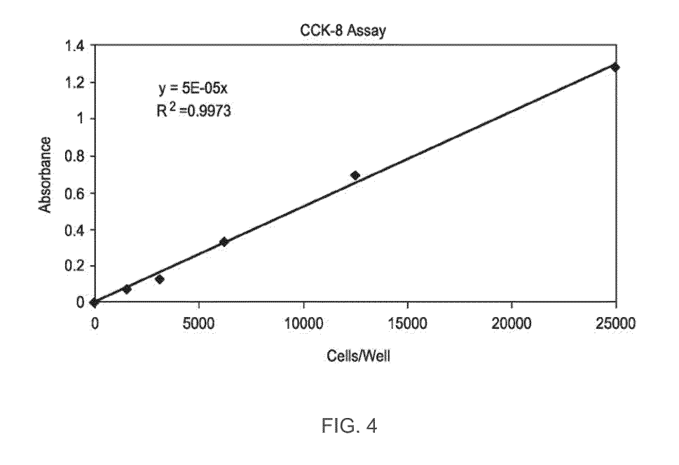

Cell count may be performed with a CCK-8 Assay. Cell Counting Kit 8 (CCK-8, Dojindo Molecular Technologies, Maryland) allows sensitive colorimetric assays for the determination of the number of viable cells in cell proliferation assays. With reference to FIG. 4, there is illustrated a standard curve of total live ASCs using the CCK-8 assay. WST-8[2-(2-methoxy-4-nitrophenyl)-3-(4-nitrophenyl)-5-(2,4-disulfophenyi)-2H-tetrazolium, monosodium salt] is reduced by dehydrogenases in cells to give a yellow colored product (formazan), which is soluble in the tissue culture medium. The amount of the formazan dye generated by the activity of dehydrogenases in cells is directly proportional to the number of living cells. The allografts were thawed and rinsed with PBS and then patted dry. Growth medium and CCK-8 solution were added into the allografts at a ratio of 10:1 cultured at 37° C. for 2 hours and evaluated in a plate reader with excitation set to 460 nm and emission set to 650 nm. The results were interpolated from a standard curve (FIG. 4) based on ASCs only (passage=3).

Histology: When the cultures were terminated, the constructs were fixed in 10% neutral buffered formal in (Sigma, St. Louis, Mo.) for 48 h, put in a processor (Citadel 2000; Thermo Shandon, Pittsburgh, Pa.) overnight, and embedded in paraffin. Sections were cut to 8 μm and mounted onto glass slides and stained with hematoxylin and eosin (H&E). Conventional light microscopy was used to analyze sections for matrix and cell morphology.

Statistical Analysis: All quantitative data were expressed as the mean±standard deviation. Statistical analysis was performed with one-way analysis of variance. A value of p<0.05 was considered statistically significant.

Results—Final Product Appearance: FIGS. 3A-3D illustrate an appearance of strips, dowels and disks. In these embodiments, all have a cortical bottom and cancellous top. Other embodiments may be used.

### b. ACS Characterization

i. Flow Cytometry Analysis—Immunophenotype of SVF

The SVF were stained with CD105, CD90 and CD73 to determine if there were significant numbers of MSC present. The immunophenotype of the stromal vascular fraction was consistent from donor to donor. The large cells (mean 3%) have the following immunophenotype and mean percentage: D7-FIB+(36%), CD105+(43%), CD90+(63%), CD73+(28%) and CD34+(62%). The small cells (mean 97%) contain only a small percentage of the markers tested and therefore could not be immunophenotyped with this method: D7-FIB (5%), CD105 (6%), CD90 (15%), CD73 (6%) and CD34 (10%). The SVF contained a significant population of CD34+ cells (Large CDC34+62% and small CD34+10%). The paucity of CD45+ cells (Large 15% and small 3%) would suggest that the SVF does not contain significant numbers of WBC (CD45+, low FSC, low SSC) or hematopoietic stem cells (CD34+, low CD45+, medium FSC, low SSC). The anti-Fibroblasts/Epithelial Cells (clone D7-FIB) antibody has been reported to be a good marker for MSC. The large cells were D7-FIB+36% and the small cells were D7-FIB+5%. CD271 should be negative on SVF cells and the large cells were CD271+10% and the small cells were CD271+0%. Following adherence of the SVF (ASCs, P1), the immunophenotype became more homogenous for both the large and small cells. The large cells (53%) have the following immunophenotype and percentage: D7-FIB+(93%), CD105+(98%), CD90+(96%) and CD73+(99%). The small cells (47%) have the following immunophenotype and percentage: D7-FIB+(77%), CD105+(75%), CD90+(58%) and CD73+(83%). The ASCs has lost CD34 marker expression (P3: large 4% and small 1%) (P1: large 8% and small 6%) and the CD45+ cells remained low (P3: large 2% and small 2%) (P1: large 3% and small 1%). This would suggest that there are few WBC (CD45+, low FSC, low SSC) or hematopoietic stem cells (CD34+, low CD45+, medium FSC, low SSC) present. The anti-Fibroblasts/Epithelial Cell (clone D7-FIB) antibody for the adherent and cultured cells showed an increased expression. The large cells were D7-FIB+93% and the small cells were D7-FIB+77%. CD271 should become positive following adherence and culture of the SVF. For P3 the large cells were CD271+4% and the small cells were CD271+1%. For P1 the large cells were CD271+27% and the small cells were CD271+3%. CD271 does not seem to be a useful marker for cultured MSC but more data is required.

ii. Estimated Mean Total Percentage of MSC

CD105 was chosen to estimate the mean total percentage of MSC; although there is no single surface marker that can discern MSC in a mixed population. For the SVF with a mean of 3% large cells, a mean of 43% CD105+ cells, the mean total percentage would be 1.3%. For the SVF with a mean of 98% small cells, a mean of 6% CD105+ cells, the mean total percentage would be 5.9%. Combining the large and small totals gives a mean total of 7.2% MSC for the SVF.

iii. In Vitro Tri-Lineage Differentiation

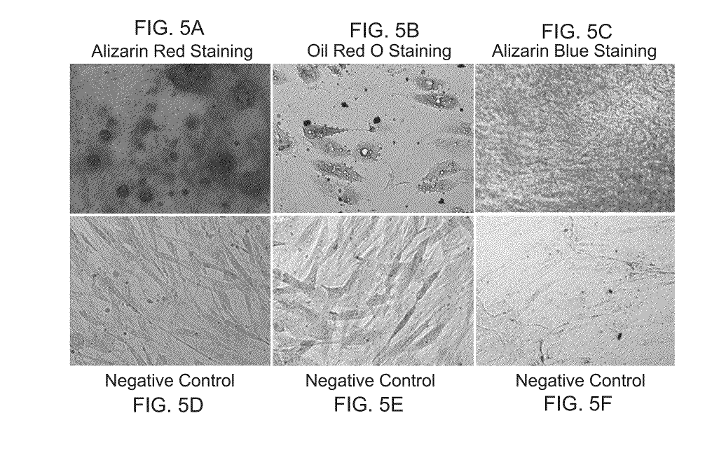

FIGS. 5A-5F illustrates mineral deposition by ASCs cultured in osteogenic medium (FIG. 5A) indicating early stages of bone formation. The samples were stained with alizarin red S. Negative controls (FIG. 5D) showed no sign of bone formation. Fat globules seen in ASCs cultured in adipogenic medium (FIG. 5B) indicating differentiation into adipocytes. The samples were stained with Oil red O. FIG. 5E shows a negative control. Proteoglycans produced by ASCs cultured in chondrogenic medium (FIG. 5C) indicating early stages of chondrogenesis. The samples were stained with alcian blue. The negative control (FIG. 5F) showed no sign of chondrogenesis.

For the osteogenic differentiation, morphological changes appeared during the second week of the culture. At the end of the 21-day induction period, some calcium crystals were clearly visible. Cell differentiation was confirmed by alizarin red staining (FIG. 5A).

The adipogenic potential was assessed by induction of confluent ASCs. At the end of the induction cycles (7 to 14 days), a consistent cell vacuolation was evident in the induced cells. Vacuoles brightly stained for fatty acid with oil red 0 staining (FIG. 5B). Chondrogenic potential was assessed by induction of confluent ASCs. At the end of the induction cycles (14 to 21 days), the induced cells were clearly different from non-induced control cells. Cell differentiation was confirmed with Alcian blue staining (FIG. 5C).

iv. Final Product Characterization

Cell count: CCK-8 Assay: 28 grafts were tested from 8 donors and had an average of 50,000 live cells/graft.

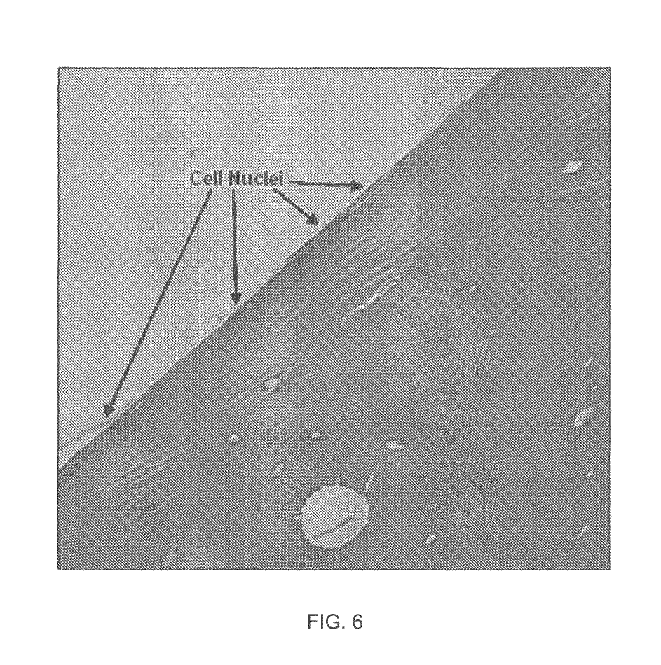

Histology: H&E was performed to demonstrate cell morphology in relation to the underlying substrate (cancellous bone matrix). The stem cells are elongated and adhere to the surface of cancellous bone. FIG. 6 is an illustration of H&E staining that showed that stem cells adhered to the bone surface.

### B. Cartilage Construct Examples

The objective was to determine whether adipose derived stem cells adhere to processed and ground articular cartilage.

ASCs adhere to cartilage, and promote cartilage repair and regeneration.

a. Experiment Design:

| Cartilage with | Cartilage w/o | | |
|---|---|---|---|
| ASCs | ASCs | ASCs only | Medium only |
| n = 3, 36 hr | n = 3, 36 hr | n = 3, 36 hr | n = 3, 36 hr |
| incubation | incubation | incubation | incubation |

b. Materials and Methods:

Sample Preparation: Cartilage pieces previously shaved from knee articulating surface and frozen at −80° C. were thawed and blended (Waring Blender) for approximately 2 minutes on “Hi” (22,000 rpms) while submerged in PBS. Resulting particles were approximately 1 mm×2-3 min×1 mm. The particles were then rinsed and drained in a sieve and were separated into six 5 ml samples and placed into a 6-well plate. Prior to seeding, cartilage samples were patted dry with sterile gauze. Three wells containing cartilage were each seeded with 200 μl cell suspension. The other three wells containing cartilage only were left as unseeded controls. An empty 6-well plate was seeded in the same fashion with three wells receiving cells and three wells without cells. The wells were incubated for an hour at 37° C. and 5% CO₂ in a humidified incubator, then submerged in 5 ml DMEM-F12/10% FBS/1% PSA and incubated for 36 hrs. All the samples in the 6-well plates were tested using CCK-8 assay for cell counts and the cartilage samples were collected for histology.

Cell count: CCK-8 Assay: Cell Counting Kit-8 (CCK-8, Dojindo Molecular Technologies, Maryland) allows sensitive colorimetric assays for the determination of the number of viable cells in cell proliferation assays. The amount of the formazan dye generated by the activity of dehydrogenases in cells is directly proportional to the number of living cells. The samples were rinsed with PBS and then patted dry. Growth medium and CCK-8 solution were added into wells at a ratio of 10:1 cultured at 37° C. for 2 hours and evaluated in a plate reader with excitation set to 460 nm and emission set to 650 nm. The results were interpolated from a standard curve based on ASCs only (passage=1).

Histology: The cartilage samples were fixed in 10% neutral buffered formalin (Sigma, St. Louis, Mo.) for 48 h, put in a processor (Citadel 2000; Thermo Shandon, Pittsburgh, Pa.) overnight, and embedded in paraffin. Sections were cut to 5 μm and mounted onto glass slides and stained with hematoxylin and eosin (H&E). Conventional light microscopy was used to analyze sections for matrix and cell morphology.

c. Results:

Cell Counts: The number of cells on cartilage was significantly different from ASCs⁻ only controls which were cultured in the 6 well plates.

| | ASCs + Cartilage | Cartilage only | Medium Only |
|---|---|---|---|
| Number of | 4,665 | 0 | 0 |
| Viable Cells | | | |

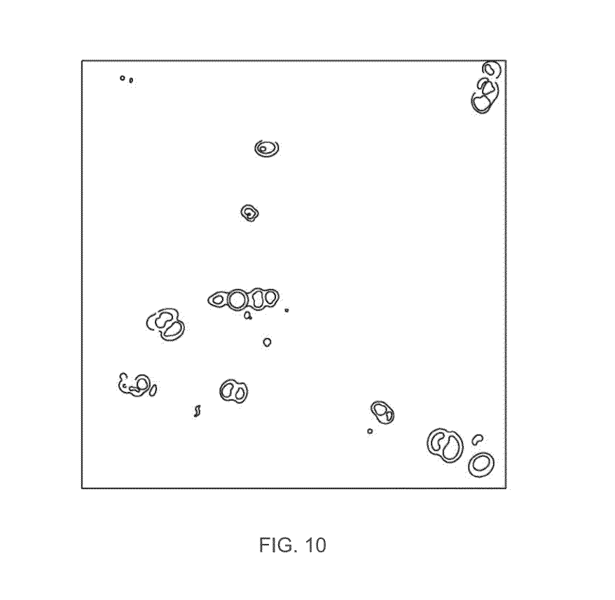

FIG. 10 illustrates H&E staining of cartilage control (10× magnification). Note that there were no live cells in the voids of the ground cartilage matrix.

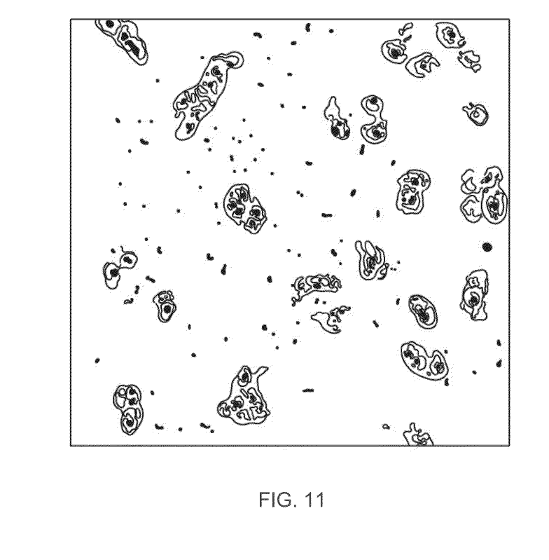

FIG. 11 illustrates H&E staining of ASCs seeded cartilage (10× magnification). Note the live cell nuclei in the voids.

In the cartilage only control, there were no live cells, only the dead cell debris was discovered. The cells seemed to be all dead and left the voids behind. In the ASCs seeded cartilage, it seemed that all the seeded cells repopulated the voids left by pre-existing cells from the cartilage. There were no live cells on the cartilage surface that lacked decellularized zones.

Conclusions: ASCs did not adhere to the cartilage matrix, however, they repopulated in the voids left from pre-existing cartilage cells.

### C. Collagen Matrix Construct Examples

### Example C1

### Adherence and Survival of Adipose-Derived Stem Cells on Acellular Dermal Matrix

### Background

Acellular dermal matrix samples were decellularized and washed in DPBS/10% PSA for 72 hours. Samples were placed in DPBS/4% PSA for 24 hours, and then placed in DPBS/1% PSA for 18 hours. Some samples to be used were placed in DMEM-F12/10% FBS/1% PSA while the rest of the tissue was stored in DPBS/4% PSA at 4° C.

### Sample Preparation

First, the acellular dermal matrix samples were removed from antibiotic storage. Next, circular samples were cut to fit snugly into 24-well plate (diameter=15.6 mm) to avoid floating, while covering the entire well bottom. There were three rinsing groups: (a) DPBS stored samples, rinsed in DPBS/1% PSA (“Group A”); (b) DPBS stored samples, rinsed in DMEM-F12/20% FBS/1% PSA (“Group B”); and (c) Media stored samples, rinsed in DMEM-F12/20% FBS/1% PSA (“Group C”). For each rinsing group, samples were placed into 125 ml vented Erlenmeyer flask with 50 ml of either DPBS/1% PSA or DMEM-F12/20% FBS/1% PSA and shaken at 37° C. in horizontal shaker for 60 minutes at 100-125 RPM. Three rinses were performed, with the reagent changed at each rinse. The samples were then removed from the flask and placed in DMEM-F12/10% FBS/1% PSA (all Groups) in 24-well plate until seeding (>10 min). The plate layouts are shown below in Table 1 and Table 2.

TABLE 1
Plate 1 layout

| | Original Well | Rinse Well | Final Well | Controls |
|---|---|---|---|---|
| Group A | Top* | 1.8 ml total | 1.8 ml total | Cells only |
| | 200,000 cells | volume | volume | 200,000 cells |
| | 1.8 ml total | | | 1.8 ml total |
| | volume | | | volume |
| | Bottom** | 1.8 ml total | 1.8 ml total | Cells only |
| | 200,000 cells | volume | volume | 200,000 cells |
| | 1.8 ml total | | | 1.8 ml total |
| | volume | | | volume |
| Group B | Top* | 1.8 ml total | 1.8 ml total | Media only |
| | 200,000 cells | volume | volume | (pre-inc) |
| | 1.8 ml total | | | 1.8 ml total |
| | volume | | | volume |
| | Bottom** | 1.8 ml total | 1.8 ml total | Media only |
| | 200,000 cells | volume | volume | (post-inc) |
| | 1.8 ml total | | | 1.8 total |
| | volume | | | volume |
| Group C | Top* | 1.8 ml total | 1.8 ml total | Top |
| | 200,000 cells | volume | volume | No cells |
| | 1.8 ml total | | | 1.8 ml total |
| | volume | | | volume |
| | Bottom** | 1.8 ml total | 1.8 ml total | Bottom |
| | 200,000 cells | volume | volume | No cells |
| | 1.8 ml total | | | 1.8 ml total |
| | volume | | | volume |

*“Top” refers to the outward epidermal facing surface or basement membrane
**“Bottom” refers to the deeper dermal or hypodermal facing surface

TABLE 2
Plate 2 layout

| | Original Well | Rinse Well | Final Well | Controls |
|---|---|---|---|---|
| Group A | Top* | 1.8 ml total | 1.8 ml total | Top |
| | 200,000 cells | volume | volume | No cells |
| | 1.8 ml total | | | 1.8 ml total |
| | volume | | | volume |
| | Bottom** | 1.8 ml total | 1.8 ml total | |
| | 200,000 cells | volume | volume | |
| | 1.8 ml total | | | |
| | volume | | | |
| Group B | Top* | 1.8 ml total | 1.8 ml total | Top |
| | 200,000 cells | volume | volume | No cells |
| | 1.8 ml total | | | 1.8 ml total |
| | volume | | | volume |
| | Bottom** | 1.8 ml total | 1.8 ml total | |
| | 200,000 cells | volume | volume | |
| | 1.8 ml total | | | |
| | volume | | | |
| Group C | Top* | 1.8 ml total | 1.8 ml total | Top |
| | 200,000 cells | volume | volume | No cells |
| | 1.8 ml total | | | 1.8 ml total |
| | volume | | | volume |
| | Bottom** | 1.8 ml total | 1.8 ml total | Bottom |
| | 200,000 cells | volume | volume | No cells |
| | 1.8 ml total | | | 1.8 ml total |
| | volume | | | volume |

*“Top” refers to the outward epidermal facing surface or basement membrane
**“Bottom” refers to the deeper dermal or hypodermal facing surface

### Seeding

Cultured adipose-derived stem cells (ASCs) were isolated by DPBS wash and TRYPLE™ Express detachment (cells used: 113712 (P1)). The cells were centrifuged and counted on Countess and diluted to 1.0×10⁶ cells/ml. The media was aspirated from all sample wells, and 200,000 cells (200 μl) were added to each sample and positive control well. The volume of all wells was gently brought up to 1.8 ml with culture media (DMEM-F12/10% FBS/1% PSA). The samples were placed in a 37° C. CO₂ incubator for 42-48 hours.

### Evaluation

For evaluating the samples, first the media was warmed to 37° C. and PRESTOBLUE™ to room temperature. The sample plates were removed from the incubator, then 1.8 1 media was added to each “Rinse” and “Final” well. With forceps, each graft was removed from the “Original” well and submerged 8-10 times in the “Rinse” well, then placed in the “Final” well, with appropriate orientation. For Plate 1 only, 200 μl of PRESTOBLUE™ reagent was added to each sample and control well. The samples were then incubated in the CO₂ incubator for 3 hours. Following incubation, seeded samples were removed to DPBS (-Ca/-Mg) in a new 12-well plate and placed in a shaker with low RPM. Triplicate aliquots were removed to black 96-well plate(s) for fluorescence reading, and the highest adherence samples (brightest readings) and no cell control were used for TRYPLE™ Express detachment and cell count. Plate 2 samples were then prepared for H&E histology using the highest adherence samples as seen from Plate 1.

### Visual Assessment

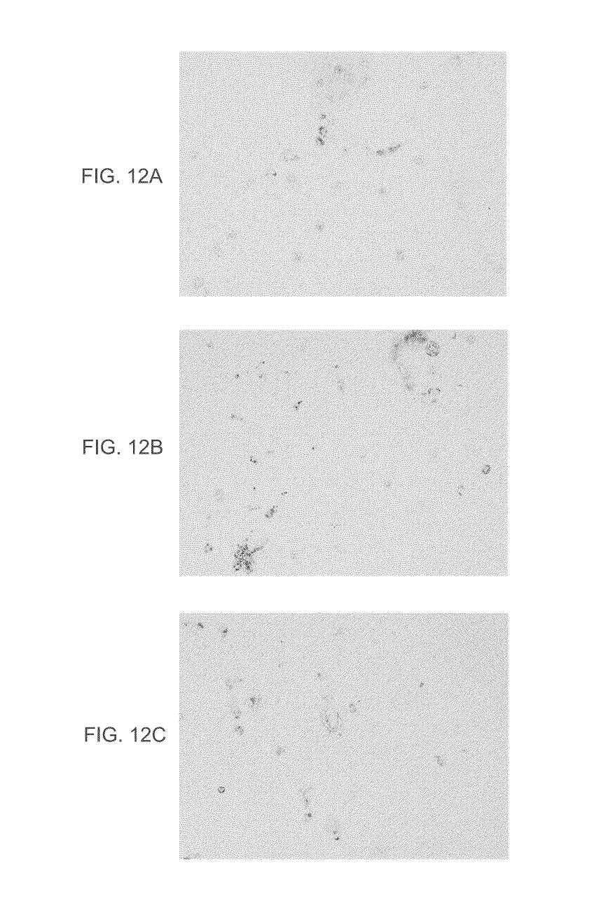

Each Original, Rinse, and Final well were viewed under inverted microscope (sample removed from Final), as shown in FIGS. 12A-12C. Group A and Group B wells were very similar for Top and Bottom samples. No live cells were visualized in any of the wells. For Group A, both top and bottom sample wells had the same general appearance, with the exception of the Top Rinse well, which had a noticeable amount of oily residue. The Group A Original and Rinse wells had small to medium amounts of dead cells and debris. The Final wells had slightly less dead cells and debris. The Group B wells were alike to Group A, with the exception of the Bottom original well, which had a noticeably larger amount of dead cells than the other wells in A or B.

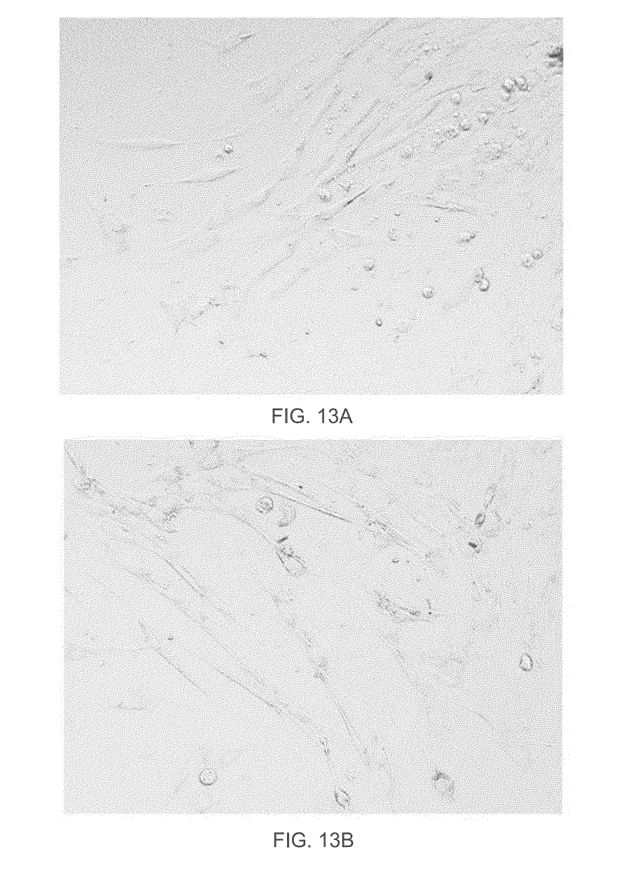

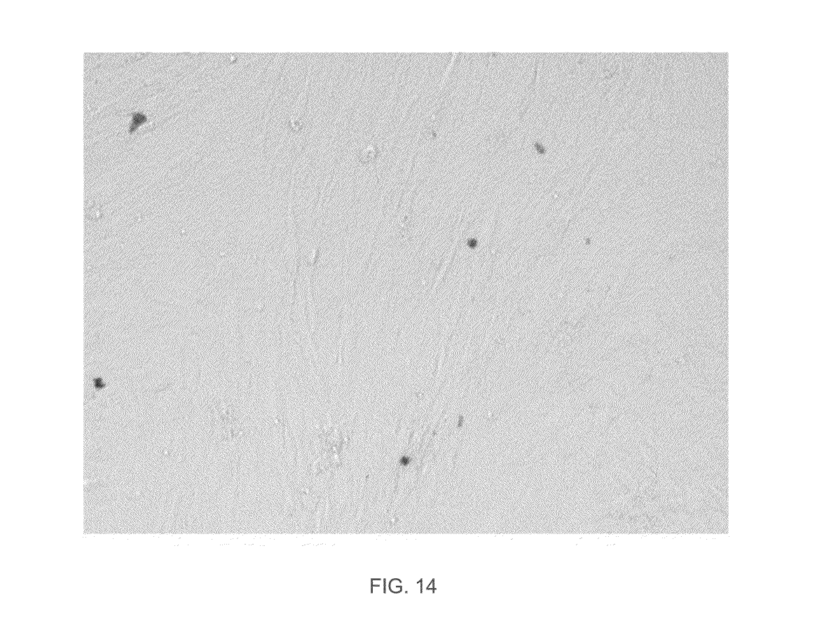

The Group C Rinse and Final wells were all similar to those in Groups A and B, showing medium amounts of dead cells and debris. However, the Group C Original wells were the only wells in any sample group to show live cells (FIGS. 13A-13B). The Top Original well had small amounts of floating dead cells in the middle with adhered living cells all around the rim. These cells likely poured over the edge of the graft and were able to adhere to the plastic during incubation. The Group C Bottom Original well also had live cells around the edges. The Bottom Original well had more visible cells than the top Original well, and this was expected because the sample had floated partially free from the plate, allowing cells to flow around. Both Group C Original wells also had medium amounts of dead cells throughout. The cell only control wells showed elongated, healthy looking cells near confluence (FIG. 14).

### PRESTOBLUE™ Metabolic Assay

The percentage of metabolic activity was compared using the fluorescence (Table 3) and absorbance (Table 4) measurements from the PRESTOBLUE™ assay, and setting the cell-only positive control as the maximum possible level of activity. Media only backgrounds were subtracted from each sample well and positive control. Each sample was compared to the positive control, and the percent of metabolic activity for each well position was recorded. (The Group C Bottom sample partially floated free from the well plate, allowing cells to flow around and adhere to the plastic.)

TABLE 3
PRESTOBLUE ™ Metabolic Assay based on fluorescence
Percentage of cells compared to control group
(Based on metabolic activity - PRESTOBLUE ™ fluorescence)

| | Original- seeded | | Final well- on |
|---|---|---|---|
| | well | Rinse well | skin |
| Group A Top | 4% | 1% | 46% |
| Group A Bottom | 4% | 0% | 25% |
| Group B Top | 5% | 2% | 60% |
| Group B Bottom | 4% | 0% | 28% |
| Group C Top | 5% | 2% | 65% |
| Group C Bottom | 59% | 1% | 45% |
| Unseeded samples | 4% | | |
| Cells only | 100% | | |

* The Group C Bottom sample partially floated free from the well plate, allowing cells to flow around and adhere to the plastic.

TABLE 4
PRESTOBLUE ™ Metabolic Assay based on absorbance
Percentage of cells compared to control group
(Based on metabolic activity - PRESTOBLUE ™ absorbance)

| | Original- seeded | | Final well- on |
|---|---|---|---|
| | well | Rinse well | skin |
| Group A Top | −12% | 2% | 39% |
| Group A Bottom | −10% | 0% | 21% |
| Group B Top | −7% | 2% | 56% |
| Group B Bottom | −6% | 1% | 25% |
| Group C Top | −4% | 4% | 69% |
| Group C Bottom | 46% | 2% | 45% |
| Unseeded samples | 1% | | |
| Cells only | 100% | | |

* The Group C Bottom sample partially floated free from the well plate, allowing cells to flow around and adhere to the plastic.

Multiple trends were apparent in the metabolic activities. The Top surface of the skin showed higher metabolic activity using PRESTOBLUE™ reagent. The cells may more readily adhere to the Top than the Bottom or they may be more metabolically active after 48 hrs on the Top surface than on the Bottom.

Another trend was that the samples that were stored and rinsed in DMEM-F12/FBS had the highest metabolic activities and presumably the highest seeding efficiency. Although all Groups had a short soak in media immediately prior to seeding, the exposure to the serum-containing media was very different for the life of the samples. Those in Group C were stored in the media and rinsed in media prior to seeding. Samples from Groups A and B were stored in DPBS. Group A was rinsed in DPBS while Group B was rinsed in media.

### TRYPLE™ Express Digestion

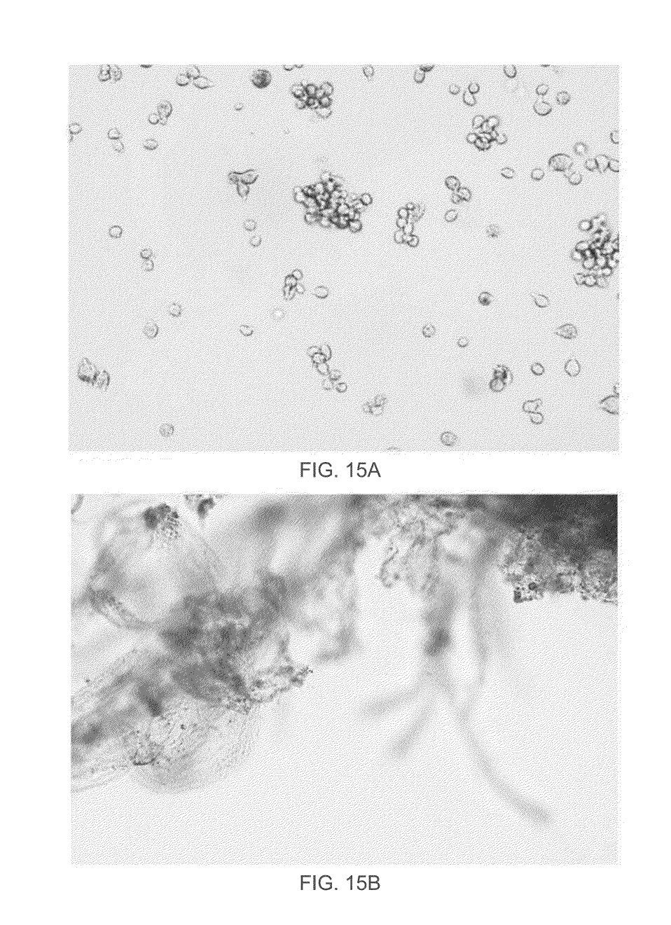

Following the PRESTOBLUE™ assay, samples from each group were washed in DPBS and cells were detached using TRYPLE™ Express. The cell populations were then centrifuged and re-plated in a 6-well plate. All seeded samples had recoverable cell populations, as viewed under the microscope. The Group C Top sample, which showed the highest level of metabolic activity, also showed the largest number of cells under the microscope, as shown in FIG. 15A. The unseeded sample (FIG. 15B) did not show any cells released.

### Example C2

### Preparation of Adipose Tissue for Forming a Cell Suspension

### Background

Adipose for generating stem cells is typically recovered as lipoaspirate using a liposuction device. However, the liposuction process is tedious and rarely results in more than 1000 cc of adipose from a typical donor. Therefore, different recovery methods such as adipose en bloc by hand were investigated to maximize the amount of tissue recovered from a single donor. En bloc adipose could yield 2 L from a single donor, thus increasing the cell yields by a factor of 2. In this study, we compared the cell counts and cell phenotype of the cells recovered using both liposuction machine and en bloc adipose from the same donor.

### Phase I: Method of Manipulation

The fibrous nature of the connective tissue within the en bloc adipose made simple manual manipulation impossible. It was determined that mechanical force was necessary for reducing particle size efficiently and consistently. The initial objective was to break down the large pieces of adipose into small particles to ensure efficient collagenase digestion.

Adipose en bloc was obtained from 2 donors and manipulated using various processing tools and food preparation devices in an attempt to prepare the tissue for collagenase digestion. The processing tools used were a meat grinder, an electric bone grinder, a meat tenderizer, a cheese grater, and a blender. The post-manipulation and post-digestion appearance of the adipose were recorded. The en bloc tissue was divided into groups and subjected to each form of manipulation. Those deemed successful at reducing particle size were then digested in collagenase and the cells were isolated.

The following methods of manipulation were successful based on ease of use, repeatability, physical appearance of manipulated adipose and resulting cell counts/viability on Countess: (1) electric bone grinder (EBG) with traditional particle set or small particle set; and (2) TSM #10 meat grinder, ⅜″ and 3/16″ pore size. In particular, the ⅜″ pore size meat grinder gave an appearance much like lipoaspirate.

### Phase II: Grinder and Tissue Washing Comparison

The manipulation of adipose en bloc was further tested, using the EBG with small particle set or prototype aggressive particle set as compared to the meat grinder using the ⅜″ pore plate or the 3/16″ pore plate. Additionally, procedures for rinsing the tissue were tested.

Adipose en bloc from an additional three donors was obtained and processed using variations of grinders and attachments as well as rinsing techniques to optimize viable cell numbers and best mimic lipoaspirate characteristics. Donor 3 was used to compare the meat grinder plate attachments (⅜″ vs 3/16″) and EBG with small particle set. Minimal variation between viable cell numbers in final pellets was found. Donor 4 was used to compare the meat grinder with ⅜″ plate and EBG with aggressive particle set, using samples from each that were rinsed pre-grinding only or rinsed pre- and post-grinding. Donor 5 was used for a verification test with the same protocol as for Donor 4.

The ⅜″ grinding plate was preferable due to ease of use and resulting similarity of the product to lipoaspirate particle size. Additionally, the speed and consistency of the meat grinder was superior to that of the EBG, although both grinders resulted in comparable numbers of viable cells. No conclusions could be drawn regarding rinsing pre-grinding only as compared to rinsing pre- and post-grinding.

### Phase III: Adipose En Bloc vs Lipoaspirate for Isolating Stromal Vascular Fraction

The purpose of this study was to isolate a cell suspension (stromal vascular fraction, or SVF) from both en bloc and liposuction adipose from the same donor and utilize flow cytometry to characterize the cell populations obtained. The samples were processed in the following ways: (1) lipoaspiration; (2) adipose en bloc with ⅜″ meat grinder plate and with pre-digestion rinse (pre-grinding and post-grinding); and (3) adipose en bloc with ⅜″ meat grinder plate and no pre-digestion rinse (pre-grinding only). Adipose from five additional donors was recovered using both liposuction and en bloc from the same donor. Liposuction adipose was recovered from the abdominal area, while en bloc adipose was recovered from the abdominal area as well as the thighs. 200 cc samples for each pathway were processed in parallel. The lipoaspirate was processed according to standard protocols, which includes draining transport media followed by three DPBS rinses in a separatory funnel before digestion with collagenase.

The adipose en bloc followed two pathways prior to collagenase digestion, after which point standard protocols were used for processing. Prior to digestion, ˜500 cc of the adipose en bloc was submerged in an equal volume of DPBS and poured back and forth between two beakers a total of six pours. This rinse was repeated for three total rinses. The adipose en bloc was then ground using the meat grinder and ⅜″ plate. The ground adipose en bloc was then divided into two 200 cc samples. One sample was rinsed three times with DPBS in the separatory funnel prior to digestion while the other sample went straight to digestion after grinding. The en bloc pathway utilizing the extra rinse may slightly increase processing time compared to the lipoaspirate pathway. However, processing without the post-grinding rinse will decrease the overall processing time as compared to lipoaspirate.

The resulting SVF samples were analyzed by flow cytometry for various cell surface markers (CD 73, 90, 105, 34, 45, 271 and D&-Fib) to test for cell viability and positive and negative mesenchymal stem cell markers.

TABLE 5
Flow cytometry analysis of SVF samples

| | | Meat | | Meat | |
|---|---|---|---|---|---|
| | | grinder | | grinder | |
| | Lipoaspirate: | en bloc + | | en bloc, no | |
| | avg | rinse: avg | | rinse: avg | |
| | cells/cc | cells/cc | ANOVA | cells/cc | ANOVA |
| | adipose* | adipose* | p-value | adipose* | p-value |
| Total | 144,713 | 105,200 | 0.411 | 175,650 | 0.598 |
| Live | 118,604 | 77,257 | 0.300 | 133,394 | 0.780 |
| CD73 | 13,004 | 13,193 | 0.984 | 24,781 | 0.278 |
| CD90 | 100,542 | 53,120 | 0.252 | 107,087 | 0.904 |
| CD105 | 32,581 | 8,263 | 0.144 | 14,769 | 0.271 |
| CD271 | 4,410 | 5,257 | 0.836 | 9,052 | 0.273 |
| D7-FIB | 30,785 | 21,324 | 0.702 | 32,363 | 0.951 |
| CD34 | 84,272 | 36,494 | 0.135 | 66,446 | 0.596 |
| CD45 | 17,673 | 21,965 | 0.692 | 31,949 | 0.177 |

*n = 5

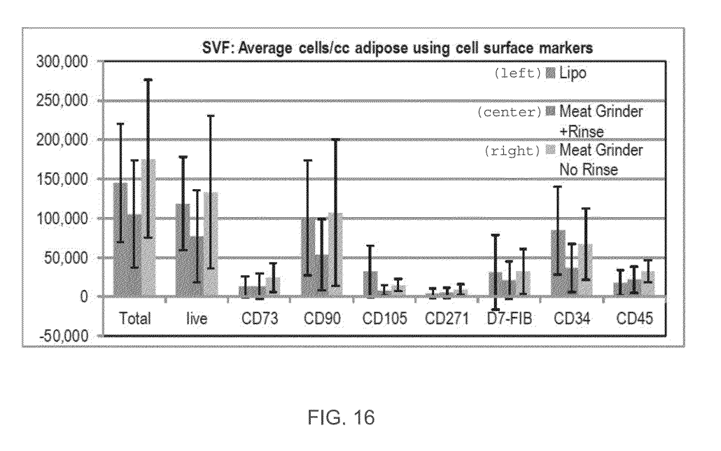

Table 5 and FIG. 16 show that there was no significant difference of live and total cell counts between lipoaspirate and meat grinder en bloc+rinse or between lipoaspirate and meat grinder en bloc no rinse. Additionally, the surface markers were not significantly different.

There were no significant differences between Lipoaspirate and either of the Meat Grinder samples for any of the categories tested. The averages showed that the largest amount of total live cells came from the meat grinder no rinse method, as did the higher averages of CD73+ and CD90+. The highest averages of CD105+ were from the lipoaspirate method. CD34+ cells were very similar between the lipoaspirate and the meat grinder no rinse methods, while CD45+ was highest in the meat grinder no rinse method and lowest in the lipoaspirate method. The meat grinder+rinse samples showed mid-range or lowest amounts for all of the categories tested. We therefore chose the meat grinder with no rinse method as the method for processing en bloc adipose.

### CONCLUSION

This study demonstrates a method of breaking down the en bloc adipose effectively for collagenase digestion. Our data also suggested that cell counts and cell phenotype per cc of adipose tissue were not significantly different between liposuction adipose and en bloc adipose. For liposuction, the volume of fat yielded per donor is 1 L, with an SVF yield/cc fat of 118,604 and a SVF yield/donor of 118 million. For en bloc processing, the volume of fat yielded per donor is 2 L, with an SVF yield/cc fat of 133,394 and a SVF yield/donor of 267 million. Therefore, en bloc adipose recovery is an effective means to increase the yield by increasing the total volume of adipose we can obtain per donor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of making an allograft composition for treating a soft tissue injury, the method comprising:
(a) providing a cell suspension comprising mesenchymal stem cells and non-mesenchymal stem cells derived from tissue obtained from a cadaveric donor;
(b) seeding the cell suspension onto an acellular collagen matrix derived from tissue obtained from the cadaveric donor;
(c) incubating the acellular collagen matrix seeded with the cell suspension under conditions suitable for adhering the mesenchymal stem cells to the acellular collagen matrix to form a seeded matrix; and
(d) rinsing the seeded matrix to remove the non-adherent cells from the seeded matrix, thereby forming the allograft composition comprising the acellular collagen matrix with mesenchymal stem cells adhered thereto.

2. The method of claim 1, wherein the acellular collagen matrix is skin, dermis, tendon, ligament, muscle, amnion, meniscus, small intestine submucosa, or bladder.

3. The method of claim 1, furthering comprising treating the collagen matrix to reduce immunogenicity prior to seeding the cell suspension.

4. The method of claim 3, wherein treating the collagen matrix to reduce immunogenicity comprises contacting the collagen matrix with a decellularizing agent.

5. The method of claim 3, wherein treating the collagen matrix to reduce immunogenicity comprises removing an epidermis layer without decellularizing the collagen matrix.

6. The method of claim 3, wherein the treated collagen matrix has at least 50% fewer endogenous cells than a corresponding untreated collaged matrix of the same type.

7. The method of claim 3, wherein the treated collagen matrix has a DNA content that is decreased by at least 50% as compared to a corresponding untreated collaged matrix of the same type.

8. The method of claim 1, wherein the collagen matrix is non-immunogenic.

9. The method of claim 1, wherein the collagen matrix comprises at least one of bioactive cytokines or bioactive growth factors.

10. The method of claim 1, wherein the cadaveric donor is human, porcine, bovine, or equine.

11. The method of claim 1, wherein the cadaveric donor is human.

12. The method of claim 1, wherein the cell suspension is derived from tissue at least one of adipose tissue, muscle tissue, birth tissue, skin tissue, bone tissue, or bone marrow tissue.

13. The method of claim 1, wherein the cell suspension is derived from adipose tissue, the cell suspension comprising a stromal vascular fraction of the adipose tissue.

14. The method of claim 1, wherein the cell suspension is derived from the tissue by digesting the tissue.

15. The method of claim 1, wherein the incubating comprises incubating the seeded matrix in growth medium.

16. The method of claim 1, wherein the incubating is performed for up to 24 hours, 36 hours, 48 hours, 60 hours, or 72 hours.

17. The method of claim 1, wherein the incubating is performed for 42-48 hours.

18. The method of claim 1, comprising placing the allograft composition into a cryopreservation medium.

19. An allograft composition comprising a combination of mesenchymal stem cells adhered to acellular dermal collagen matrix, the allograft composition manufactured by the method of claim 1.

20. A method of treating a soft tissue injury in a subject, the method comprising administering the allograft composition of claim 19 to the site of the soft tissue injury.

21. The method of claim 58, wherein the composition is administered topically.

22. The method of claim 58, wherein the composition is administered by surgical implantation.
